(19) United States
(12) Patent Application Publication
(10) Pub. No.: US 2025/0205330 A1
(43) Pub. Date: Jun. 26, 2025

(54) METHODS FOR PROVIDING PROTECTION TO PORCINE EPIDEMIC DIARRHEA VIRUS (PEDV) WITH A PLANT PRODUCED VACCINE

(71) Applicant: Mazen Animal Health, Inc., Ames, IA (US)

(72) Inventors: John Howard, Cayucos, CA (US); Erin Egelkrout, San Luis Obispo, CA (US); Celine Hayden, San Louis Obispo, CA (US)

(73) Assignee: Mazen Animal Health, Inc., Ames, IA (US)

(21) Appl. No.: 18/989,993

(22) Filed: Dec. 20, 2024

Related U.S. Application Data
(60) Provisional application No. 63/613,292, filed on Dec. 21, 2023.

(51) Int. Cl. A61K 39/225; A61K 9/00; C07K 14/165

(52) U.S. Cl. CPC A61K 39/225; A61K 9/0053; C07K 14/165

(57) ABSTRACT

Methods for producing a protective immune response to Porcine Epidemic Diarrhea Virus (PEDV) is provided wherein a fusion protein comprising the S1 Spike protein of the virus operably fused to the heat labile enterotoxin B subunit (LTB) peptide is expressed in a plant and the plant or plant product comprising the fusion protein is orally administered to an animal. The vaccine can be produced by introducing into a plant a construct comprising a promoter preferentially directing expression to seed of said plant, a nucleic acid encoding the S1-LTB fusion protein and a nucleic acid targeting expression to the endoplasmic reticulum of the plant. When orally administered to an animal, a protective response is observed, including a serum antibody response and mucosal immune response.

### CROSS REFERENCE TO RELATED APPLICATIONS

This application claims priority to U.S. Provisional Application No. 63/613,292, filed Dec. 21, 2023, which is herein incorporated by reference in its entirety.

### SEQUENCE LISTING

The instant application contains a Sequence Listing which has been submitted in XML format via USPTO Patent Center and is hereby incorporated by reference in its entirety. Said XML copy, created on Dec. 21, 2023 is named M117885_1060US.P1_0022.8_ST26.xml and is 85,213 bytes in size.

### SUMMARY

Methods for producing a protective immune response to Porcine Epidemic Diarrhea Virus (PEDV) is provided wherein a fusion protein comprising the S1 Spike protein of the virus operably fused to the heat labile enterotoxin B subunit (LTB) peptide is expressed in a plant and the plant or plant product comprising the fusion protein is orally administered to an animal. The vaccine can be produced by introducing into a plant a construct comprising a promoter preferentially directing expression to seed of said plant, a nucleic acid encoding the S1-LTB fusion protein and a nucleic acid targeting expression to the endoplasmic reticulum of the plant. When orally administered to an animal, a protective response is observed, including a serum antibody response and mucosal immune response.

Embodiments of the invention include:
1. A method of producing a protective response to Porcine Epidemic Diarrhea Virus (PEDV) in an animal, the method comprising,
a) orally administering to said animal a composition comprising plant or plant product comprising a S1-LTB fusion protein, wherein said S1-LTB fusion protein comprises the Spike (S1) protein of PEDV operably fused to the heat labile enterotoxin B subunit (LTB) peptide, wherein said S1 protein comprises SEQ ID NO: 3, 4, 9, 21 or 22 or a sequence having at least 90% identity to SEQ ID NO: 3, 4, 9, 21 or 22 or a functional fragment of said S1 protein; and wherein said LTB peptide comprises SEQ ID NO: 15 or a sequence having at least 90% identity to SEQ ID NO: 15 or a functional fragment of said LTB peptide; and
b) producing a protective response to said PEDV in said animal.
2. The method of embodiment 1, wherein said protective response comprises a serum antibody response in said animal.
3. The method of embodiment 2, wherein said serum antibody response is at least 20 times greater than serum antibody response in an animal not administered said vaccine.
4. The method of any one of embodiments 1-3, wherein said composition comprising said S1-LTB fusion protein elicits a greater mucosal immune response than a composition comprising said S1 protein without said LTB peptide operably fused thereto.
5. The method of embodiment 4, wherein said greater mucosal immune response comprises greater levels of fecal anti-PEDV immunoglobulin A (IgA) in said animal.
6. The method of any one of embodiments 1-5, wherein said composition comprising said S1-LTB fusion protein comprises a higher ratio of high molecular weight S1 protein to low molecular weight S1 protein in comparison to a composition comprising said S1 protein without said LTB peptide operably fused thereto.
7. A method of producing a greater mucosal immune response to Porcine Epidemic Diarrhea Virus (PEDV) in an animal, the method comprising,
a) orally administering to said animal a composition comprising plant or plant product comprising a S1-LTB fusion protein, wherein said S1-LTB fusion protein comprises the Spike (S1) protein of PEDV operably fused to the heat labile enterotoxin B subunit (LTB) peptide, wherein said S1 protein comprises SEQ ID NO: 3, 4, 9, 21 or 22 or a sequence having at least 90% identity to SEQ ID NO: 3, 4, 9, 21 or 22 or a functional fragment of said S1 protein; and wherein said LTB peptide comprises SEQ ID NO: 15 or a sequence having at least 90% identity to SEQ ID NO: 15 or a functional fragment of said LTB peptide; and
b) producing a mucosal immune response to said PEDV in said animal, wherein said mucosal immune response in said animal is greater in comparison to an animal that has been administered a composition comprising said S1 protein without said LTB peptide operably fused thereto.
8. The method of embodiment 7, wherein said mucosal immune response comprises an increase in fecal anti-PEDV immunoglobulin A (IgA), and wherein levels of fecal anti-PEDV IgA are higher in an animal administered a composition comprising said S1-LTB fusion protein in comparison to an animal administered a composition comprising said S1 protein without said LTB peptide operably fused thereto.
9. The method of embodiment 7 or 8, wherein said composition comprising said S1-LTB fusion protein comprises a higher ratio of high molecular weight S1 protein to low molecular weight S1 protein in comparison to a composition comprising said S1 protein without said LTB peptide operably fused thereto.
10. The method of any one of embodiments 1-9, wherein milk of said animal comprises said S1-LTB fusion protein.
11. The method of any one of embodiments 1-10, wherein said composition comprises plant material or plant tissue.
12. The method of any one of embodiments 1-10, wherein said composition comprises seed or embryo of seed.
13. The method of any one of embodiments 1-12, wherein said animal is a pig.
14. The method of any one of embodiments 1-13, wherein said S1 protein comprises SEQ ID NO: 3, 4, 9, 21 or 22 or a sequence having at last 95% identity thereto.
15. The method of any one of embodiments 1-14, wherein said S1 protein comprises SEQ ID NO: 3, 4, 9, 21 or 22 or a functional fragment thereof.
16. The method of any one of embodiments 1-15, wherein said LTB peptide comprises SEQ ID NO: 15 or a sequence having at least 95% identity to SEQ ID NO: 15.
17. The method of any one of embodiments 1-16, wherein said LTB peptide comprises SEQ ID NO: 15.
18. The method of any one of embodiments 1-17, wherein said S1-LTB fusion protein further comprises at least one of the COE peptide of SEQ ID NO: 12 and the DC3 peptide of SEQ ID NO: 13 operably fused thereto.
19. The method of any one of embodiments 1-18, wherein said plant or plant product comprises a construct comprising a nucleic acid molecule encoding said S1-LTB fusion protein operably linked to:
a) a promoter preferentially directing expression to seed tissue of a plant; and
b) a nucleic acid molecule targeting expression of said S1-LTB fusion protein in the endoplasmic reticulum of said plant.
20. The method of embodiment 19, wherein said construct comprises two copies of said nucleic acid molecule encoding said S1-LTB fusion protein.
21. The method of embodiment 19 or 20, wherein said S1 protein is encoded by a nucleotide sequence comprising SEQ ID NO: 1 or 2, or a sequence having at least 90% identity to SEQ ID NO: 1 or 2.
22. The method of any one of embodiments 19-21, wherein said S1 protein is encoded by a sequence comprising SEQ ID NO: 1, 2 or 25 or a sequence having at least 95% identity thereto.
23. The method of any one of embodiments 19-22, wherein said S1 protein is encoded by the nucleotide sequence comprising SEQ ID NO: 1, 2, or 25.
24. The method of any one of embodiments 19-23, wherein said LTB peptide is encoded by a nucleotide sequence comprising SEQ ID NO: 14, or a sequence having at least 90% identity to SEQ ID NO: 14.
25. The method of any one of embodiments 19-24, wherein said LTB peptide is encoded by a nucleotide sequence comprising SEQ ID NO: 14, or a sequence having at least 95% identity to SEQ ID NO: 14.
26. The method of any one of embodiments 19-25, wherein said LTB peptide is encoded by the nucleotide sequence comprising SEQ ID NO: 14.

### BRIEF DESCRIPTION OF THE DRAWINGS

FIG. 1 is a graphic showing constructs created for introduction into plants. Promoters used were pr25, pr39 and pr44. BAASS refers to the barley alpha amylase sequence, S1 refers to the US strain spike proteins where S1ext(US) refers to an extended sequence and S1(DR13) refers to a South Korean strain spike protein; Vac refers to a vacuole targeting sequence, KDEL refers to an endoplasmic reticulum retaining sequence; COE refers to the COE sequence; PinII is the PinII terminator and LTB refers to the heat labile enterotoxin subunit, all of which are described in further detail here.

FIG. 2 is a gel of a Western blot, showing expression of the S protein.

FIG. 3 is a gel of a Western blot of S1 expression in representative seed.

FIG. 4 is a graph showing pig antibody response after exposure to oral or injected vaccines. Group 1 is the injection group, Group 2 is the oral vaccination group and Group 3 is the control, as described below.

FIG. 5 is a gel of a Western blot of PDK seed extracts using anti-S or anti-LTB antibodies.

FIG. 6 is a graph showing the levels of S1 protein in fractions from a size exclusion chromatography column of extracts from maize engineered to express S1 protein (PDC) or S1 protein fused to the heat labile enterotoxin B subunit LTB (PDK).

FIG. 7 is a graph showing levels of anti-PEDV IgA in fecal samples from pigs injected with a commercial vaccine (INJ), orally administered control corn (CON), orally administered a PDC vaccine (PDC), or orally administered a PDK vaccine (PDK).

### BACKGROUND

The porcine epidemic diarrhea virus is an enveloped, positive-sense single-stranded RNA virus that causes acute diarrhea, vomiting, and dehydration in pigs. It was first identified in Europe but has become increasingly problematic in many Asian countries, including Korea, China, Japan, the Philippines, and Thailand. In April of 2013, PEDV emerged in U.S. swine in the Midwest, swiftly spreading across the country. By October 2013, PEDV was detected in swine herds in 18 States. The economic impact of PEDV infection has already been substantial. North American isolates of PEDV have been identified (Huang, et al. 2013; Stevenson et al. 2013), however no fully licensed vaccine is commercially available in the United States. Accordingly, there is a continuing need to develop vaccines capable of protecting pigs against disease associated with PEDV.

### DESCRIPTION

Porcine Epidemic Diarrhea Virus (PEDV) is a member of the subfamily Coronavirinae of genus Alphacoronavirus (Bridgen et al. 1993 Sequence determination of the nucleocapsid protein gene of the porcine epidemic diarrhea virus confirms that this virus is a coronavirus related to human coronavirus 229E and porcine transmissible gastroenteritis virus. J. Gen. Virol. 74 (Pt 9):1795-1804) and was first identified in England in 1971 and later in other countries, such as Belgium, China, Hungary, Italy, Japan, Korea, and Thailand (Oldham J. 1972 Letter to the editor. Pig Farming 1972 (October suppl):72-73; Pensaert and De Bouck P. 1978 A new coronavirus-like particle associated with diarrhea in swine. Arch. Virol. 58:243-247; Molecular characterization and phylogenetic analysis of membrane protein genes of porcine epidemic diarrhea virus isolates in China. Virus Genes 36:355-364; Nagy et al. 1996. Enterotoxigenic Escherichia coli, rotavirus, porcine epidemic diarrhea virus, adenovirus and calici-like virus in porcine postweaning diarrhea in Hungary. Acta Vet. Hung. 44:9-19; Martelli et al. 2008. Epidemic of diarrhea caused by porcine epidemic diarrhea virus in Italy. Vet. Rec. 162:307-310; Takahashi et al. 1983. An outbreak of swine diarrhea of a new-type associated with coronavirus-like particles in Japan. Nippon Juigaku Zasshi 45:829-832; Chae et al. 2000. Prevalence of porcine epidemic diarrhea virus and transmissible gastroenteritis virus infection in Korean pigs. Vet. Rec. 147:606-608; Puranaveja et al. 2009. Chinese-like strain of porcine epidemic diarrhea virus, Thailand. Emerg. Infect. Dis. 15:1112-1115). Other members of this family include Porcine Respiratory Coronavirus (PRCV), Hemagglutinating Encephalomyelitis Coronavirus (PHE), and Transmissible Gastroenteritis Virus (TGEV). Although PEDV and TGEV viruses are related and the clinical signs are very similar, there is no immune cross-protection.

PEDV is an enveloped virus possessing approximately a 28 kb, positive-sense, single stranded RNA genome, with a 5′ cap and a 3′ polyadenylated tail. (Pensaert and De Bouck P. 1978). The genome comprises a 5′ untranslated region (UTR), a 3′ UTR, and at least seven open reading frames (ORFs) that encode four structural proteins (spike (S), envelope (E), membrane (M), and nucleocapsid (N)) and three non-structural proteins (replicases 1a and 1b and ORF3); these are arranged on the genome in the order 5′-replicase (1a/1b)-S-ORF3-E-M-N-3′ (Oldham J. 1972; and Bridgen et al. 1993). The first three emergent North American PEDV genomic sequences characterized, Minnesota MN (GenBank: KF468752.1), Iowa IA1 (GenBank: KF468753.1), and Iowa IA2 (GenBank: KF468754.1), have the same size of 28,038 nucleotides (nt), excluding the polyadenosine tail and share the genome organization with the prototype PEDV CV777 strain (GenBank: AF353511.1). These three North American PEDV sequences shared 99.8 to 99.9% nucleotide identities. In particular, strains MN and IA2 had only 11 nucleotide differences across the entire genome.

Coronavirus particles display large surface projections comprised of homotrimers of the spike (S) glycoproteins. The PEDV Spike (S) protein is a type I glycoprotein composed of about 1,383 amino acids (aa) (1386 with the Korea strain, see e.g., GenBank Ref NO. AAM19716.1 (SEQ ID NO: 22), identifying the S1 region as residues 234-736; 1382 amino acids in a China strain, with S1 identified as 230-732, see GenBank Ref No. AFL02627.1 (SEQ ID NO: 23)). It contains a putative signal peptide (residues 1-24). The S protein can be divided into two regions. One is the N-terminal region of S1 (1-733 or 735 aa). Referring to the Spike protein used in the example below, it has 94% identity to Korean strain example of AAM19716.1 and 93% identity with the China strain example of AFL02627.1. The other region is the C-terminal region S2 which is identified as including residues 736-741 to the end of the Spike protein based on its homology with S protein of other coronaviruses (Chang et al. 2002 Identification of the epitope region capable of inducing neutralizing antibodies against the porcine epidemic diarrhea virus. Mol. Cells 14, 295-299. Cleavage of the spike protein into S1 and S2 can occur in the presence of trypsin. (See e.g., Wicht et al. (2014) Proteolytic activation of the porcine epidemic diarrhea coronavirus spike fusion protein by trypsin in cell culture. J. Virol. 88:2952-7961). The GPRLQPY motif located at the carboxy-terminal of the spike protein induces antibodies that neutralize Porcine epidemic diarrhea virus. Godet et al. 1994 Virus Res. 132, 192-196. Major receptor-binding and neutralization determinants are located within the same domain of the transmissible gastroenteritis virus (coronavirus) spike protein. J. Virol. 68, 8008-8016; Jackwood et al. 2001. Spike glycoprotein cleavage recognition site analysis of infectious bronchitis virus. Avian Dis. 45, 366-372; Sturman et al. 1984 Proteolytic cleavage of peplomeric glycoprotein E2 of MHV yields two 90K subunits and activates cell fusion. Adv. Exp. Med. Biol. 173, 25-35. 33; Sun et al. 2008. Identification of two novel B cell epitopes on porcine epidemic diarrhea virus spike protein. Vet. Microbiol. 131, 73-81. 34.). The S protein in coronaviruses is a surface antigen, where it plays a role in regulating interactions with host cell receptor glycoproteins to mediate viral entry and stimulating induction of neutralizing antibodies in the natural host. A phylogenetic and genetic comparison analysis of the S gene and its regions showed minor variations among strains, including the US, China, Korea, showed a percent identity ranging from 89.4% to 100% identity. This included percent identity of comparison of strains DR13, BM1, J3142, BM3, CV777, AH2012, BJ-2012-2, Colorado30, Indiana34 and Texas 31. See Chung et al (2017) Genetic characterization of S1 domain of porcine epidemic diarrhea viruses spike proteins isolated in Korea, J. Immune Disord. Vol. 1 No. 1. Sequence comparisons of the polypeptide of the S protein showed Korean isolates had 93.6% to 99.6% identity with each other and 92.2%-93.7% identity with other strains. Lee et al. (2010) Hetergeneity in spike protein genes of porcine epidemic diarrhea viruses isolate din Kore, Virus Res. 149(2):175-82. Thus, the S glycoprotein is a primary target for the development of effective vaccines against PEDV.

The PEDV M protein is the most abundant envelope component playing an important role in the viral assembly process and also induces antibodies that neutralize the virus. Likewise, the PEDV N protein, which binds to virion RNA providing a structural basis for the nucleocapsid, may also be important for induction of cell-mediated immunity (Saif, L. 1993 Coronavirus immunogens. Vet. Microbiol. 285-297).

The only accessory gene in PEDV is ORF3. While accessory genes are generally maintained in field strains, alteration of ORF3 is thought to influence virulence; cell culture adaptation has been used to alter the ORF3 gene in order to reduce virulence (Song et al. 2003 Differentiation of a Vero cell adapted porcine epidemic diarrhea virus from Korean field strains by restriction fragment length polymorphism analysis of ORF 3. Vaccine 21, 1833-1842). In fact, through investigation of the ORF3 gene, researchers have charted the emergence of new genogroups of PEDV in immunized swine herds in China since 2006. Phylogenic studies of these strains and the geographical reemergence of PEDV in China have demonstrated that those field strains causing devastating enteric disease differ genetically in ORF3 from the European strains and vaccine strains (Park et al. 2011) Molecular characterization and phylogenetic analysis of porcine epidemic diarrhea virus (PEDV) field isolates in Korea. Arch. Virol. 156, 577-585.

Different strains of PEDV exist with varying levels of virulence. The clinical signs of PEDV infection are similar to transmissible gastroenteritis virus (TGEV) infection (Pijpers et al. 1993). In pigs three weeks of age and younger, clinical signs (including acute watery, diarrhea, vomiting, and dehydration) can be seen as soon as 24 hours after PEDV infection leading to 100% mortality. PEDV-infected feeder and grower pigs, as well as sows and boars, can develop diarrhea and vomiting. The animals can also show signs of anorexia and can be lethargic. Older pigs show reduced feed efficiency, additional days to market, and the susceptibility of infected animals to secondary infections is likely. For sows, reduced body condition may negatively impact reproductive performance.

The gross and histological changes in the gut of animals infected with PEDV are similar in the United States as those observed in China; essentially the virus destroys the villi of a pig's intestine so that there is a failure to absorb nutrients. PED virus infects and replicates in the cells lining the small and large intestine. Viral replication causes vacuolization and destruction of epithelial cells, leading to villous atrophy and enteritis. Approximately 36 hours after infection, neonatal pigs suffer a severe loss of absorptive epithelium, causing malabsorption, diarrhea, and death of 50-100% of infected piglets. Animals succumbing to the disease in the Minnesota and Iowa outbreaks had gross pathological lesions confined to the small intestine and the small intestine was characterized by thin translucent walls distended with yellow fluid. Histological evaluations revealed regions of small intestines with villus blunting and fusion and minimal lymphoblastic infiltration of the villi of the lamia propria. The induction of mucosal immune responses (such as secretion of IgA against PEDV) can be used as a method to protect against PEDV infection.

Huang et al. 2013 characterized three different strains of PEDV from outgoing outbreaks in the United States—one from Minnesota and two from Iowa, designated MN (GenBank accession No: KF468752) and IA1 (GenBank accession No: KF468753) and IA2 (GenBank accession No: KF48754), respectively. (Huang et al. 2013 Origin, evolution, and genotyping of emergent porcine epidemic diarrhea virus strains in the United States. mBio 4(5):e00737-13.) Huang's phylogenic survey grouped PEDV strains as falling into two distinct genogroups, designated genogroup 1 (G1) and genogroup 2 (G2). Genogroup 1 includes at least three clusters 1a, 1b, and R. Subgroup 1a includes the early European, Chinese, and Korean isolates, e.g., prototype CV777 strain (Belgium, 1978, GenBank: AF353511.1) and strains LZC (Gansu, China, 2006; GenBank: EF185992) and SM98 (Korea, 1998; GenBank: GU937797.1). Subgroup 1b contains five strains—one from South Korea (the DR13 attenuated vaccine strain, GenBank: JQ023162.1) and the others from China linked by the common “genetic signature” 8-aa deletion in nsp3 and the large ORF3 deletion at the C terminus. Group “R” is associated with recombinants of the other genogroups. Certain strains belong to genogroup G2a. The Chinese strain AH2012 (GenBank accession no: KC210145) and the North American strains share several unique nucleotides changes and are clustered together in genogroup 2a. Nucleotide identity to AH2012 for strains MN and IA2 was 99.6% and for strain IA1 was 99.5%. A closely related North American isolate US/Colorado/2013 (GenBank Accession No: KF272920.1) has also been reported by Marthaler et al, 2013 Complete genome sequence of porcine epidemic diarrhea virus strain USA/Colorado/2013 from the United States. Genome Announc. 1(4):e00555-13.10.1128/genomeA.00555-13. Like the North American isolates above, the complete PEDV genome of CO/13 has a nucleotide identity of 96.5 to 99.5% with other complete PEDV genomes available in GenBank, with the highest nucleotide identity (99.5%) with Chinese strain AH2012 (GenBank Accession No. KC210145). It is a member of the 2a genogroup.

Attempts to create PEDV vaccines include production of attenuated viral vaccines, such as that described at U.S. Pat. No. 9,950,061, incorporated by reference in its entirety. The attenuated vaccine included a Spike antigen, with that modified Spike protein shown as sequence identifier 9, encoded by the nucleic acid of sequence identifier 8 with variations effective for protection shown having at least 80% homology and included sequence identifiers 3, 7, 9 and 14, all of which are incorporated by reference herein in their entirety.

Here is provided a plant-produced Spike (S) polypeptide and a vaccine for PEDV comprising the same, wherein the S1 protein is operably fused to the heat labile enterotoxin B (LTB) subunit peptide or an active fragment or variant thereof. In an embodiment the S polypeptide fused to LTB is introduced into a plant in a construct comprising a seed-preferred promoter which may further prefer expression to the embryo of the seed, operably linked to the nucleic acid molecule encoding the S polypeptide-LTB fusion. In further embodiments the construct comprises nucleic acid molecules that retain expression of the S polypeptide-LTB fusion in the endoplasmic reticulum of the cell of the plant. Still further embodiments provide for two plant transcription units (PTUs) with each PTU comprising an embryo preferred promoter and nucleic acid molecules retaining expression in the endoplasmic reticulum. Additional embodiments provide the PTUs comprising the same seed preferred promoter and nucleic acid molecules retaining expression in the endoplasmic reticulum.

The S protein is expressed poorly in recombinant systems, therefore, it is difficult to develop a commercial subunit vaccine. Here in an embodiment, maize grain is used as a basis for the production of the subunit vaccine. High expression levels of at least 10 mg/kg of whole seed are obtained. An embodiment provides for a range of about 10-100 mg/kb. Further embodiments provide for expression at 11 mg/kb, 12 mg/kg, 13, 14, 15, 16, 17, 18, 19, 20, 25, 30, 35, 40 mg/kg of whole seed or more or amounts in-between.

The presently disclosed compositions and methods utilize vaccines comprising a plant, plant part or a product produced from the plant, such as a seed, grain, flour or other edible composition comprising the plant, plant part or product produced therefrom comprising the PEDV S1 protein operably fused to the non-toxic subunit of the heat-labile toxin from E. coli (LTB). LTB binds to gangliosides present on cell surfaces. This presents points of recognition for extracellular molecules that might serve to enhance the immune response upon oral administration.

In some embodiments, compositions comprising S1-LTB fusion proteins comprise a greater ratio of high molecular weight S1 protein to low molecular weight S1 protein when compared to compositions comprising the S1 protein without a LTB fused thereto. Without being bound by any theory or mechanism of action, it is believed that these high molecular weight compositions are comprised of polymers of the S1 protein, such as dimers or trimers, which might lead to an enhanced immune response given that the polymeric compositions produced with the S1-LTB fusions more closely mimic the homotrimers on the surface of the PEDV virus. High molecular weight S1 protein compositions comprise a weight that is more than the weight of a single, non-polymeric S1 protein or S1-LTB fusion protein. Low molecular weight S1 protein compositions comprise a weight that is approximately equal to or less than the weight of a single, non-polymeric S1 protein or S1-LTB fusion protein. Compositions comprising S1-LTB fusion proteins can comprise a ratio of high molecular weight S1 protein to low molecular weight S1 protein that is within the range of 1.1 to 10-fold more than the control (i.e., compositions comprising S1 protein without a LTB fused thereto), including but not limited to about 1.1-fold, about 1.2-fold, about 1.3-fold, about 1.4-fold, about 1.5-fold, about 2-fold, about 2.25-fold, about 2.5-fold, about 2.75-fold, about 3-fold, about 3.25-fold, about 3.5-fold, about 3.75-fold, about 4-fold, about 4.25-fold, about 4.5-fold, about 4.75-fold, about 5-fold, about 5.25-fold, about 5.5-fold, about 5.75-fold, about 6-fold, about 6.25-fold, about 6.5-fold, about 6.75-fold, about 7-fold, about 7.25-fold, about 7.5-fold, about 7.75-fold, about 8-fold, about 8.25-fold, about 8.5-fold, about 8.75-fold, about 9-fold, about 9.25-fold, about 9.5-fold, about 9.75-fold, about 10-fold-fold, or more. Any method known in the art to measure the amount of S1 protein of various molecular weights can be used, including but not limited to size exclusion chromatography followed by an ELISA of column fractions such as described herein. Oral administration of the plant, plant part or a product produced from the plant part, such as a seed, grain, flour or other edible composition comprising the plant, plant part or product produced therefrom comprising the Spike protein results in a surprising serum response from animals, and can also produce a mucosal response as well. Further, oral administration of the plant, plant part or a product produced from the plant part, such as a seed, grain, flour or other edible composition comprising the plant, plant part or product produced therefrom comprising the Spike-LTB fusion protein results in a strong mucosal response. The serum or mucosal response in an embodiment is within the range of two-100 fold more than the control. In another embodiment the response can be 5 times, 10 times, 15 times, 20 times, 25 times, 30 times, 35 times, 40 times, 45 times, 50 times, 55 times, 60 times, 65 times, 70 times, 75 times, 80 times, 85 times, 90 times, 95 times or more greater than control animals not receiving vaccination, or amounts in-between.

Moreover, oral administration of the plant, plant part or a product produced from the plant part, such as a seed, grain, flour or other edible composition comprising the plant, plant part or product produced therefrom comprising the Spike protein-LTB fusion results in a greater mucosal immune response from animals in comparison to a vaccine comprising a Spike protein without a LTB fused thereto. The mucosal immune response in an embodiment is within the range of two-100 fold more than the control (i.e., a Spike protein without a LTB fused thereto). In another embodiment the response can be 5 times, 10 times, 15 times, 20 times, 25 times, 30 times, 35 times, 40 times, 45 times, 50 times, 55 times, 60 times, 65 times, 70 times, 75 times, 80 times, 85 times, 90 times, 95 times or more greater than control animals, or amounts in-between. wherein said composition comprising said S1-LTB fusion protein produces higher molecular weight S1 protein compositions in comparison to a composition comprising said S1 protein without said LTB peptide operably fused thereto.

As used herein, the terms nucleic acid or polynucleotide refer to deoxyribonucleotides or ribonucleotides and polymers thereof in either single- or double-stranded form. The sequence used to make the vaccine may be obtained from any source, such as a biological source in isolating from a biological sample or can refer to a sequence synthetically produced based upon the sequence obtained from the sample. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Furthermore, the terms are used herein to include naturally-occurring nucleic acid molecules, which can be isolated from a cell, as well as synthetic molecules, which can be prepared, for example, by methods of chemical synthesis or by enzymatic methods such as by the polymerase chain reaction (PCR). Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

Nucleic acids employed here include those that encode an entire S1-LTB fusion polypeptide as well as those that encode a subsequence of the fusion polypeptide or produce a fragment that provides a protective response. For example, nucleic acids that encode a fusion polypeptide which is not full-length but nonetheless has protective activity against PEDV or produces a greater mucosal immune response than a S1 protein or active variant or fragment thereof alone. The invention includes not only nucleic acids that include the nucleotide sequences as set forth herein, but also nucleic acids that are substantially identical to, correspond to, or substantially complementary to, the exemplified embodiments. For example, the invention includes nucleic acids that include a nucleotide sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%, 94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The nucleotide sequence may be modified as described previously, so long as any polypeptide encoded by and produced is capable of inducing the generation of a protective response.

The nucleic acids can be obtained using methods that are known to those of skill in the art. Suitable nucleic acids (e.g., cDNA, genomic, or subsequences) can be cloned, or amplified by in vitro methods such as the polymerase chain reaction (PCR) using suitable primers, the ligase chain reaction (LCR), the transcription-based amplification system (TAS), or the self-sustained sequence replication system (SSR). A wide variety of cloning and in vitro amplification methodologies are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (2001) Molecular Cloning—A Laboratory Manual (Third ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Amheim & Levinson (Oct. 1, 1990) C& EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039. Nucleic acids or subsequences of these nucleic acids, can be prepared by any suitable method as described above, including, for example, cloning and restriction of appropriate sequences.

“Codon optimization” can be used to optimize sequences for expression in an animal and is defined as modifying a nucleic acid sequence for enhanced expression in the cells of the animal of interest, e.g. swine, by replacing at least one, more than one, or a significant number, of codons of the native sequence with codons that are more frequently or most frequently used in the genes of that animal. Various species exhibit particular bias for certain codons of a particular amino acid.

As used herein, a “polypeptide” refers generally to peptides and proteins. In certain embodiments the polypeptide may be at least two, three, four, five, six, seven, eight, nine or ten or more amino acids or more or any amount in-between. A peptide is generally considered to be more than fifty amino acids. The terms “fragment,” “derivative” and “homologue” when referring to the polypeptides according to the present invention, means a polypeptide which retains essentially the same biological function or activity as said polypeptide, that is, act as an antigen and/or provide treatment for and/or protection against disease. Such fragments, derivatives and homologues can be chosen based on the ability to retain one or more of the biological activities of the polypeptide, that is, act as an antigen and/or provide treatment for and/or protection against the pathogen. The polypeptide vaccines of the present invention may be recombinant polypeptides, natural polypeptides or synthetic polypeptides, preferably recombinant polypeptides. One skilled in the art appreciates that it is possible that the protective polypeptide may be expressed by the gene in the host cells and the plant composition administered to the animal or extracted from the plant prior to administration.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode a protective polypeptide are preferably optimized for expression in a particular host cell (e.g., yeast, mammalian, plant, fungal, and the like) used to produce the polypeptide or RNA.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton & Lange, Norwalk, Conn. (1994). Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The isolated variant proteins can be purified from cells that naturally express it, purified from cells that have been altered to express it (recombinant), or synthesized using known protein synthesis methods. For example, a nucleic acid molecule encoding the variant polypeptide is cloned into an expression vector, the expression vector introduced into a host cell and the variant protein expressed in the host cell. The variant protein can then be isolated from the cells by an appropriate purification scheme using standard protein purification techniques. Many of these techniques are described in detail below.

The methods include amino acids that include an amino acid sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%, 94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The sequence may be modified as described previously, so long the polypeptide is capable of inducing the generation of a protective response.

The presently disclosed vaccines comprise fusion proteins wherein a S1 protein is attached to heterologous sequences. Such chimeric and fusion proteins comprise a protein operably fused (i.e., the coding sequences of the S1 protein and the heterologous protein are ligated together in-frame, such that both proteins are produced) to a heterologous protein having an amino acid sequence not substantially homologous to the variant protein, such as the enterotoxin B subunit (LTB) peptide. The heterologous protein can be fused to the N-terminus or C-terminus of the S1 protein.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A variant protein-encoding nucleic acid can be cloned into such an expression vector such that the fusion moiety is linked in-frame to the variant protein.

Polypeptides sometimes contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art. Accordingly, the variant peptides of the present invention also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The present methods further provide functional fragments of the nucleic acid molecules and polypeptides including variant proteins of the polypeptide, in addition to proteins and peptides that comprise and consist of such fragments, provided that such fragments act as an antigen and/or provide treatment for and/or protection against PEDV.

As used herein, the term “subunit” refers to a portion of the microorganism which provides protection and may itself be antigenic, i.e., capable of inducing an immune response in an animal. The term should be construed to include subunits which are obtained by both recombinant and biochemical methods.

In one embodiment, a method of identifying protective sequences of the virus or nucleic acids that elicit protection is provided. This method also includes fragments, derivatives, or homologs of the nucleic acid molecule. In one aspect, the method comprises administering to a test animal such sequences. The test and control animals are subsequently challenged with an infectious amount of a microorganism that causes the disease. Various methods and techniques for determining whether protection is provided are known to those skilled in the art, including but not limited to, observing a difference between the test and control animal in the symptoms of the disease, for example. A decrease in any of the symptoms observed in the test animal compared to the control animal indicates that protective molecule(s) provide a degree of protection against disease. Similar symptoms or an increase in any of the symptoms observed in the test animal compared to those observed in the control animal indicate that the protective molecule(s) do not provide protection.

In another aspect, determining whether the molecules provided protection against PEDV includes determining the presence or absence of challenge disease in the test animal by electron microscopy or antibody or assays such as the fluorescent focusing neutralizing (FFN) test or Western blot assay may be used. PCR methods may be used to determine if the protective molecule is present. Northern blotting can detect the presence of diagnostic sequences. In another aspect, an ELISA or similar assay, such as a hemagglutinin inhibition assay are the types of many varied assays that can determine if the protective molecule is effective. The ELISA or enzyme linked immunoassay has been known since 1971. In general, antigens solubilized in a buffer are coated on a plastic surface. When serum is added, antibodies can attach to the antigen on the solid phase. The presence or absence of these antibodies can be demonstrated when conjugated to an enzyme. Adding the appropriate substrate will detect the amount of bound conjugate which can be quantified. A common ELISA assay is one which uses biotinylated anti-(protein) polyclonal antibodies and an alkaline phosphatase conjugate. For example, an ELISA used for quantitative determination of protein levels can be an antibody sandwich assay, which utilizes polyclonal rabbit antibodies obtained commercially. The antibody is conjugated to alkaline phosphatases for detection. In another example, an ELISA assay to detect trypsin or trypsinogen uses biotinylated anti-trypsin or anti-trypsinogen polyclonal antibodies and a streptavidin-alkaline phosphatase conjugate.

Clearly, many such methods are available to one skilled in the art to ascertain if the molecule provides protection and provides protection at the levels administered to the animal.

The nucleic acid molecule, polypeptide or fragment thereof, when administered to the animal produces a protective response to PEDV. A protective response is elicited in the animal. The animal may or may not produce antibodies in response, but the animal will have decreased morbidity or mortality resulting from administration of the vaccine, and as described further herein. The terms “protecting”, “protection”, “protective immunity” or “protective immune response,” as used herein, are intended to mean that the host animal mounts an active immune response to the vaccine or polypeptides of the present invention, such that upon exposure to disease challenge, the animal is able to combat the infection. Thus, a protective immune response will decrease the incidence of morbidity and mortality from exposure to the microorganism among a host animal. The animal will be protected from subsequent exposure to the disease-causing agent. In an embodiment, the animal may be protected by treating the animal which has already been exposed to the disease-causing agent by administration of the vaccine or polypeptide after such exposure. In such an instance there is also shown to be a lessening of morbidity and mortality. Those skilled in the art will understand that in a commercial animal setting, the production of a protective immune response may be assessed by evaluating the effects of vaccination on the herd as a whole, e.g., there may still be morbidity and mortality in a minority of vaccinated animals. Furthermore, protection also includes a lessening in severity of any gross or histopathological changes and/or of symptoms of the disease, as compared to those changes or symptoms typically caused by the isolate in similar animals which are unprotected (i.e., relative to an appropriate control). Thus, a protective immune response will decrease the symptoms of the disease compared to a control pig.

In some embodiments, compositions comprising a plant or plant product comprising a S1-LTB fusion protein elicit a greater mucosal immune response to PEDV in an animal than a composition comprising a plant or plant product comprising a S1 protein without a LTB protein fused thereto. A mucosal immune response is one that occurs in the mucosa-associated lymphoid tissue (MALT), such as the gut-associated lymphoid tissue. Given that the dominant antibody isotype of the mucosal immune system is IgA, the mucosal immune response to a given antigen can be quantitated by measuring the levels of IgA that recognize the antigen in mucosal secretions, such as feces. In some embodiments, compositions comprising a plant or plant product comprising a S1-LTB fusion protein can elicit levels of fecal anti-PEDV IgAs that are about 1.1 to about 20-fold higher than compositions comprising a plant or plant product comprising a S1 protein without the LTB protein fused thereto, including about 1.1-fold, about 1.5-fold, about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, and about 10-fold, or more. A “construct” is a package of genetic material inserted into the genome of a cell via various techniques. A “vector” is any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA or RNA replication in vivo, i.e., capable of replication under its own control. The term “vector” includes both viral and nonviral means for introducing the nucleic acid into a cell in vitro, ex vivo or in vivo. Viral vectors include alphavirus, retrovirus, adeno-associated virus, pox, baculovirus, vaccinia, herpes simplex, Epstein-Barr, rabies virus, vesicular stomatitis virus, and adenovirus vectors. Non-viral vectors include, but are not limited to plasmids, liposomes, electrically charged lipids (cytofectins), DNA- or RNA protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest or produces RNA, and the cassette and restriction sites are designed to ensure insertion of the cassette in the proper reading frame for transcription and translation.

A nucleic acid molecule is introduced into a cell when it is inserted in the cell. A cell has been “transfected” by exogenous or heterologous DNA or RNA when such DNA or RNA has been introduced inside the cell.

A cell has been “transformed” by exogenous or heterologous DNA or RNA when the transfected DNA or RNA effects a phenotypic change. The transforming DNA can be integrated (covalently linked) into chromosomal DNA making up the genome of the cell.

Once the gene is engineered to contain desired features, such as the desired subcellular localization sequences, it may then be placed into an expression vector by standard methods. The selection of an appropriate expression vector will depend upon the method of introducing the expression vector into host cells. A typical expression vector contains prokaryotic DNA elements coding for a bacterial origin of replication and an antibiotic resistance gene to provide for the growth and selection of the expression vector in the bacterial host; a cloning site for insertion of an exogenous DNA sequence; eukaryotic DNA elements that control initiation of transcription of the exogenous gene; and DNA elements that control the processing of transcripts, such as transcription termination/polyadenylation sequences. It also can contain such sequences as are needed for the eventual integration of the vector into the host chromosome.

By “promoter” is meant a regulatory region of DNA capable of regulating the transcription of a sequence linked thereto. It usually comprises a TATA box capable of directing RNA polymerase II to initiate RNA synthesis at the appropriate transcription initiation site for a particular coding sequence. The promoter is the minimal sequence sufficient to direct transcription in a desired manner. The term “regulatory region” is also used to refer to the sequence capable of initiating transcription in a desired manner.

A nucleic acid molecule may be used in conjunction with its own or another promoter. In one embodiment, a selection marker a nucleic acid molecule of interest can be functionally linked to the same promoter. In another embodiment, they can be functionally linked to different promoters. In yet third and fourth embodiments, the expression vector can contain two or more genes of interest that can be linked to the same promoter or different promoters. For example, one promoter can be used to drive a nucleic acid molecule of interest and the selectable marker, or a different promoter used for one or each. These other promoter elements can be those that are constitutive or sufficient to render promoter-dependent gene expression controllable as being cell-type specific, tissue-specific or time or developmental stage specific, or being inducible by external signals or agents. Such elements may be located in the 5′ or 3′ regions of the gene. Although the additional promoter may be the endogenous promoter of a structural gene of interest, the promoter can also be a foreign regulatory sequence. Promoter elements employed to control expression of product proteins and the selection gene can be any host-compatible promoters. These can be plant gene promoters, such as, for example, the ubiquitin promoter (European patent application no. 0 342 926); the promoter for the small subunit of ribulose-1,5-bis-phosphate carboxylase (ssRUBISCO) (Coruzzi et al., 1984; Broglie et al., 1984); or promoters from the tumor-inducing plasmids from Agrobacterium tumefaciens, such as the nopaline synthase, octopine synthase and mannopine synthase promoters (Velten and Schell, 1985) that have plant activity; or viral promoters such as the cauliflower mosaic virus (CaMV) 19S and 35S promoters (Guilley et al., 1982; Odell et al., 1985), the figwort mosaic virus FLt promoter (Maiti et al., 1997) or the coat protein promoter of TMV (Grdzelishvili et al., 2000). Alternatively, plant promoters such as heat shock promoters for example soybean hsp 17.5-E (Gurley et al., 1986); or ethanol-inducible promoters (Caddick et al., 1998) may be used. See International Patent Application No. WO 91/19806 for a review of illustrative plant promoters suitably employed.

A promoter can additionally comprise other recognition sequences generally positioned upstream or 5′ to the TATA box, referred to as upstream promoter elements, which influence the transcription initiation rate. It is recognized that having identified the nucleotide sequences for a promoter region, it is within the state of the art to isolate and identify further regulatory elements in the 5′ region upstream from the particular promoter region identified herein. Thus, the promoter region is generally further defined by comprising upstream regulatory elements such as those responsible for tissue and temporal expression of the coding sequence, enhancers and the like.

Tissue-preferred promoters can be utilized to target enhanced transcription and/or expression within a particular tissue. When referring to preferential expression, what is meant is expression at a higher level in the particular tissue than in other tissue. Examples of these types of promoters include seed preferred expression such as that provided by the phaseolin promoter (Bustos et al. (1989) The Plant Cell Vol. 1, 839-853). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, an Ltp1 (See, for example, U.S. Pat. No. 7,550,579), an Ltp2 (Opsahl-Sorteberg, H-G. et al., (2004) Gene 341:49-58 and U.S. Pat. No. 5,525,716), and oleosin genes. See also WO 00/12733, where seed-preferred promoters from end1 and end2 genes are disclosed. Seed-preferred promoters also include those promoters that direct gene expression predominantly to specific tissues within the seed such as, for example, the endosperm-preferred promoter of 7-zein, the cryptic promoter from tobacco (Fobert et al. (1994) “T-DNA tagging of a seed coat-specific cryptic promoter in tobacco” Plant J. 4: 567-577), the P-gene promoter from corn (Chopra et al. (1996) “Alleles of the maize P gene with distinct tissue specificities encode Myb-homologous proteins with C-terminal replacements” Plant Cell 7:1149-1158, Erratum in Plant Cell 1997, 1:109), the globulin-1 promoter from corn (Belanger and Kriz (1991) “Molecular basis for Allelic Polymorphism of the maize Globulin-1 gene” Genetics 129: 863-972 and GenBank accession No. L22344), promoters that direct expression to the seed coat or hull of corn kernels, for example the pericarp-specific glutamine synthetase promoter (Muhitch et al., (2002) “Isolation of a Promoter Sequence From the Glutamine Synthetase₁₋₂ Gene Capable of Conferring Tissue-Specific Gene Expression in Transgenic Maize” Plant Science 163:865-872 and GenBank accession number AF359511) and to the embryo (germ) such as that disclosed at U.S. Pat. No. 7,169,967. When referring to a seed or an embryo preferred promoter is meant that it expresses an operably linked sequence to a higher degree in seed or embryo tissue that in other plant tissue. It may express during seed or embryo development, along with expression at other stages, may express strongly during seed or embryo development and to a much lesser degree at other times.

The range of available promoters includes inducible promoters. An inducible regulatory element is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the actin of a pathogen or disease agent such as a virus. A cell containing an inducible regulatory element may be exposed to an inducer by externally applying the inducer to the cell or plant such as by spraying, watering, heating or similar methods.

Any inducible promoter can be used. See Ward et al. Plant Mol. Biol. 22: 361-366 (1993). Exemplary inducible promoters include ecdysone receptor promoters, U.S. Pat. No. 6,504,082; promoters from the ACE1 system which responds to copper (Mett et al. PNAS 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)) Tet repressor from Tn10 (Gatz et al., Mol. Gen. Genet. 227: 229-237 (1991); or from a steroid hormone gene, the transcriptional activity of which is induced by a glucocorticosteroid hormone, Schena et al., Proc. Natl. Acad. Sci. U.S.A. 88: 10421 (1991); the maize GST promoter, which is activated by hydrophobic electrophilic compounds that are used as pre-emergent herbicides; and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Other components of the vector may be included, also depending upon intended use of the gene. Examples include selectable markers, targeting or regulatory sequences, stabilizing or leader sequences, introns etc. General descriptions and examples of plant expression vectors and reporter genes can be found in Gruber, et al., “Vectors for Plant Transformation” in Method in Plant Molecular Biology and Biotechnology, Glick et al eds; CRC Press pp. 89-119 (1993). The selection of an appropriate expression vector will depend upon the host and the method of introducing the expression vector into the host. The expression cassette will also include at the 3′ terminus of the heterologous nucleotide sequence of interest, a transcriptional and translational termination region functional in plants.

The expression vector can optionally also contain a signal sequence located between the promoter and the gene of interest and/or after the gene of interest. A signal sequence is a nucleotide sequence, translated to give an amino acid sequence, which is used by a cell to direct the protein or polypeptide of interest to be placed in a particular place within or outside the eukaryotic cell. Many signal sequences are known in the art. See, for example Becker et al., (1992) Plant Mol. Biol. 20:49, Knox, C., et al., “Structure and Organization of Two Divergent Alpha-Amylase Genes from Barley”, Plant Mol. Biol. 9:3-17 (1987), Lerner et al., (1989) Plant Physiol. 91:124-129, Fontes et al., (1991) Plant Cell 3:483-496, Matsuoka et al., (1991) Proc. Natl. Acad. Sci. 88:834, Gould et al., (1989) J. Cell. Biol. 108:1657, Creissen et al., (1991) Plant J. 2:129, Kalderon, et al., (1984) “A short amino acid sequence able to specify nuclear location,” Cell 39:499-509, Steifel, et al., (1990) “Expression of a maize cell wall hydroxyproline-rich glycoprotein gene in early leaf and root vascular differentiation” Plant Cell 2:785-793. When targeting the protein to the cell wall use of a signal sequence is necessary. One example is the barley alpha-amylase signal sequence. Rogers, J. C. (1985) “Two barley alpha-amylase gene families are regulated differently in aleurone cells” J. Biol. Chem. 260: 3731-3738.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, Zea mays Brittle-1 chloroplast transit peptide (Nelson et al. Plant Physiol 117(4):1235-1252 (1998); Sullivan et al. Plant Cell 3(12):1337-48; Sullivan et al., Planta (1995) 196(3):477-84; Sullivan et al., J. Biol. Chem. (1992) 267(26):18999-9004) and the like. One skilled in the art will readily appreciate the many options available in expressing a product to a particular organelle. Use of transit peptides is well known (e.g., see U.S. Pat. Nos. 5,717,084; 5,728,925). A protein may be targeted to the endoplasmic reticulum of the plant cell. This may be accomplished by use of a localization sequence, such as KDEL. This sequence (Lys-Asp-Glu-Leu) contains the binding site for a receptor in the endoplasmic reticulum. (Munro et al., (1987) “A C-terminal signal prevents secretion of luminal ER proteins.” Cell. 48:899-907. There are a wide variety of endoplasmic reticulum retention signal sequences available to one skilled in the art and the KDEL sequence is one example. Another example is HDEL (His-Asp-Glu-Leu (SEQ ID NO: 24)). See, for example, Kumar et al. which discuses methods of producing a variety of endoplasmic reticulum proteins. Kumar et al. (2017) “prediction of endoplasmic reticulum resident proteins using fragmented amino acid composition and support vector machine” Peer J. doi: 10.7717/peerj.3561.

Retaining the protein in the vacuole is another example. Signal sequences to accomplish this are well known. For example, Raikhel U.S. Pat. No. 5,360,726 shows a vacuole signal sequence as does Warren et al at U.S. Pat. No. 5,889,174. Vacuolar targeting signals may be present either at the amino-terminal portion, (Holwerda et al., (1992) The Plant Cell, 4:307-318, Nakamura et al., (1993) Plant Physiol., 101:1-5), carboxy-terminal portion, or in the internal sequence of the targeted protein. (Tague et al., (1992) The Plant Cell, 4:307-318, Saalbach et al. (1991) The Plant Cell, 3:695-708). Additionally, amino-terminal sequences in conjunction with carboxy-terminal sequences are responsible for vacuolar targeting of gene products (Shinshi et al. (1990) Plant Molec. Biol. 14:357-368).

The termination region can be native with the promoter nucleotide sequence, can be native with the DNA sequence of interest, or can be derived from another source. Convenient termination regions are available from the Ti-plasmid of A. tumefaciens, such as the octopine synthase (MacDonald et al., (1991) Nuc. Acids Res. 19(20)5575-5581) and nopaline synthase termination regions (Depicker et al., (1982) Mol. and Appl. Genet. 1:561-573 and Shaw et al. (1984) Nucleic Acids Research Vol. 12, No. 20 pp 7831-7846 (nos). Examples of various other terminators include the pin II terminator from the protease inhibitor II gene from potato (An, et al. (1989) Plant Cell 1, 115-122. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Many variations on the promoters, selectable markers, signal sequences, leader sequences, termination sequences, introns, enhancers and other components of the vector are available to one skilled in the art.

The term “plant” refers to the entire plant or plant material or plant part or plant tissue or plant cell including a collection of plant cells. It is used broadly herein to include any plant at any stage of development, or to part of a plant, including a plant cutting, a plant cell culture, a plant organ, a plant seed, and a plantlet. Plant seed parts, for example, include the pericarp or kernel, the embryo or germ, and the endoplasm. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. In an embodiment, the tissue culture will preferably be capable of regenerating plants. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, plants may be regenerated from the tissue cultures.

Any plant species may be used, whether monocotyledonous or dicotyledonous, including but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats (Avena), barley (Hordeum), vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers which may be employed in practicing the present invention include, for example, pines such as loblolly pine (Pinus taeda), slash pine (Pinus elliotii), ponderosa pine (Pinus ponderosa), lodgepole pine (Pinus contotta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). An embodiment provides the plant is maize.

The method of transformation/transfection is not critical; various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed.

Methods for introducing expression vectors into plant tissue available to one skilled in the art are varied and will depend on the plant selected. Procedures for transforming a wide variety of plant species are well known and described throughout the literature. (See, for example, Miki and McHugh (2004) Biotechnol. 107, 193-232; Klein et al. (1992) Biotechnology (N Y) 10, 286-291; and Weising et al. (1988) Annu. Rev. Genet. 22, 421-477). For example, the DNA construct may be introduced into the genomic DNA of the plant cell using techniques such as microprojectile-mediated delivery (Klein et al. 1992, supra), electroporation (Fromm et al., 1985 Proc. Natl. Acad. Sci. USA 82, 5824-5828), polyethylene glycol (PEG) precipitation (Mathur and Koncz, 1998 Methods Mol. Biol. 82, 267-276), direct gene transfer (WO 85/01856 and EP-A-275 069), in vitro protoplast transformation (U.S. Pat. No. 4,684,611), and microinjection of plant cell protoplasts or embryogenic callus (Crossway, A. (1985) Mol. Gen. Genet. 202, 179-185). Agrobacterium transformation methods of Ishida et al. (1996) and also described in U.S. Pat. No. 5,591,616 are yet another option. Co-cultivation of plant tissue with Agrobacterium tumefaciens is a variation, where the DNA constructs are placed into a binary vector system (Ishida et al., 1996 Nat. Biotechnol. 14, 745-750). The virulence functions of the Agrobacterium tumefaciens host will direct the insertion of the construct into the plant cell DNA when the cell is infected by the bacteria. See, for example, Fraley et al. (1983) Proc. Natl. Acad. Sci. USA, 80, 4803-4807. Agrobacterium is primarily used in dicots, but monocots including maize can be transformed by Agrobacterium. See, for example, U.S. Pat. No. 5,550,318. In one of many variations on the method, Agrobacterium infection of corn can be used with heat shocking of immature embryos (Wilson et al. U.S. Pat. No. 6,420,630) or with antibiotic selection of Type II callus (Wilson et al., U.S. Pat. No. 6,919,494).

Rice transformation is described by Hiei et al. (1994) Plant J. 6, 271-282 and Lee et al. (1991) Proc. Nat. Acad. Sci. USA 88, 6389-6393. Standard methods for transformation of canola are described by Moloney et al. (1989) Plant Cell Reports 8, 238-242. Corn transformation is described by Fromm et al. (1990) Biotechnology (N Y) 8, 833-839 and Gordon-Kamm et al. (1990) supra. Wheat can be transformed by techniques similar to those used for transforming corn or rice. Sorghum transformation is described by Casas et al. (Casas et al. (1993). Transgenic sorghum plants via microprojectile bombardment. Proc. Natl. Acad. Sci. USA 90, 11212-11216) and barley transformation is described by Wan and Lemaux (Wan and Lemaux (1994) Generation of large numbers of independently transformed fertile barley plants. Plant Physiol. 104, 37-48). Soybean transformation is described in a number of publications, including U.S. Pat. No. 5,015,580.

In one method, the Agrobacterium transformation methods of Ishida et al. (1996) and also described in U.S. Pat. No. 5,591,616, are generally followed, with modifications that the inventors have found improve the number of transformants obtained. The Ishida method uses the A188 variety of maize that produces Type I callus in culture. In an embodiment the Hi II maize line is used which initiates Type II embryogenic callus in culture (Armstrong et al., 1991).

While Ishida recommends selection on phosphinothricin when using the bar or pat gene for selection, another preferred embodiment provides use of bialaphos instead. In general, as set forth in the 5,591,616 patent, and as outlined in more detail below, dedifferentiation is obtained by culturing an explant of the plant on a dedifferentiation-inducing medium for not less than seven days, and the tissue during or after dedifferentiation is contacted with Agrobacterium having the gene of interest. The cultured tissue can be callus, an adventitious embryo-like tissue or suspension cells, for example. In this preferred embodiment, the suspension of Agrobacterium has a cell population of 10⁶ to 10¹¹ cells/ml and are contacted for three to ten minutes with the tissue, or continuously cultured with Agrobacterium for not less than seven days. The Agrobacterium can contain plasmid pTOK162, with the gene of interest between border sequences of the T region of the plasmid, or the gene of interest may be present in another plasmid-containing Agrobacterium. The virulence region may originate from the virulence region of a Ti plasmid or Ri plasmid. The bacterial strain used in the Ishida protocol is LBA4404 with the 40 kb super binary plasmid containing three vir loci from the hypervirulent A281 strain. The plasmid has resistance to tetracycline. The cloning vector cointegrates with the super binary plasmid. Since the cloning vector has an E. coli specific replication origin, but not an Agrobacterium replication origin, it cannot survive in Agrobacterium without cointegrating with the super binary plasmid. Since the LBA4404 strain is not highly virulent, and has limited application without the super binary plasmid, the inventors have found in yet another embodiment that the EHA101 strain is preferred. It is a disarmed helper strain derived from the hypervirulent A281 strain. The cointegrated super binary/cloning vector from the LBA4404 parent is isolated and electroporated into EHA101, selecting for spectinomycin resistance. The plasmid is isolated to assure that the EHA101 contains the plasmid. EHA101 contains a disarmed pTi that carries resistance to kanamycin. See, Hood et al. (1986).

Further, the Ishida protocol as described provides for growing fresh culture of the Agrobacterium on plates, scraping the bacteria from the plates, and resuspending in the co-culture medium as stated in the 5,591,616 patent for incubation with the maize embryos. This medium includes 4.3 g MS salts, 0.5 mg nicotinic acid, 0.5 mg pyridoxine hydrochloride, 1.0 ml thiamine hydrochloride, casamino acids, 1.5 mg 2,4-D, 68.5 g sucrose and 36 g glucose per liter, all at a pH of 5.8. In a further preferred method, the bacteria are grown overnight in a 1 ml culture and then a fresh 10 ml culture is re-inoculated the next day when transformation is to occur. The bacteria grow into log phase and are harvested at a density of no more than OD₆₀₀=0.5, preferably between 0.2 and 0.5. The bacteria are then centrifuged to remove the media and resuspended in the co-culture medium. Since Hi II is used, medium preferred for Hi II is used. This medium is described in considerable detail by Armstrong and Green (1985). The resuspension medium is the same as that described above. All further Hi II media are as described in Armstrong and Green (1985). The result is redifferentiation of the plant cells and regeneration into a plant. Redifferentiation is sometimes referred to as dedifferentiation, but the former term more accurately describes the process where the cell begins with a form and identity, is placed on a medium in which it loses that identity and becomes “reprogrammed” to have a new identity. Thus, the scutellum cells become embryogenic callus.

A transgenic plant may be produced that contains an introduced nucleic acid molecule encoding the polypeptide.

When referring to introduction of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. This can be accomplished by any means known in the art for breeding plants such as, for example, cross pollination of the transgenic plants that are described above with other plants, and selection for plants from subsequent generations which express the amino acid sequence. The plant breeding methods used herein are well known to one skilled in the art. For a discussion of plant breeding techniques, see Poehlman (1995) Breeding Field Crops. AVI Publication Co., Westport Conn, 4^{th} Edit). Many crop plants useful in this method are bred through techniques that take advantage of the plant's method of pollination. A plant is self-pollinating if pollen from one flower is transferred to the same or another flower of the same plant. A plant is cross-pollinating if the pollen comes from a flower on a different plant. For example, in Brassica, the plant is normally self-sterile and can only be cross-pollinated unless, through discovery of a mutant or through genetic intervention, self-compatibility is obtained. In self-pollinating species, such as rice, oats, wheat, barley, peas, beans, soybeans, tobacco and cotton, the male and female plants are anatomically juxtaposed. During natural pollination, the male reproductive organs of a given flower pollinate the female reproductive organs of the same flower. Maize plants (Zea mays L.) can be bred by both self-pollination and cross-pollination techniques. Maize has male flowers, located on the tassel, and female flowers, located on the ear, on the same plant. It can self or cross-pollinate.

Pollination can be by any means, including but not limited to hand, wind or insect pollination, or mechanical contact between the male fertile and male sterile plant. For production of hybrid seeds on a commercial scale in most plant species pollination by wind or by insects is preferred. Stricter control of the pollination process can be achieved by using a variety of methods to make one plant pool male sterile, and the other the male fertile pollen donor. This can be accomplished by hand detassling, cytoplasmic male sterility, or control of male sterility through a variety of methods well known to the skilled breeder. Examples of more sophisticated male sterility systems include those described by Brar et al., U.S. Pat. Nos. 4,654,465 and 4,727,219 and Albertsen et al., U.S. Pat. Nos. 5,859,341 and 6,013,859.

Backcrossing methods may be used to introduce the gene into the plants. This technique has been used for decades to introduce traits into a plant. An example of a description of this and other plant breeding methodologies that are well known can be found in references such as Neal (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

Selection and propagation techniques described above can yield a plurality of transgenic plants that are harvested in a conventional manner. The plant or any parts expressing the recombinant polypeptide can be used in a commercial process, or the polypeptide extracted. When using the plant or part itself, it can, for example, be made into flour and then applied in the commercial process. Polypeptide extraction from biomass can be accomplished by known methods. Downstream processing for any production system refers to all unit operations after product synthesis, in this case protein production in transgenic seed (Kusnadi, A. R., Nikolov, Z. L., Howard, J. A., 1997. Biotechnology and Bioengineering. 56:473-484). For example, seed can be processed either as whole seed ground into flour or, fractionated and the germ separated from the hulls and endosperm. If germ is used, it is usually defatted using an extraction process and the remaining crushed germ ground into a meal or flour. In some cases, the germ is used directly in the process or the protein can be extracted (See, e.g. WO 98/39461). Extraction is generally made into aqueous buffers at specific pH to enhance recombinant protein extraction and minimize native seed protein extraction. Subsequent protein concentration or purification can follow.

The compositions and process described here are also to producing and administering a vaccine that protects an animal from PEDV.

As used herein, the term “vaccine” as used herein refers to a pharmaceutical composition comprising at least one protective molecule, that induces a protective response in an animal and possibly, but not necessarily, one or more additional components that enhance the activity of said active component. A vaccine may additionally comprise further components typical to pharmaceutical compositions. In another form, the immunologically active component of a vaccine may comprise appropriate elements of said organisms (subunit vaccines) whereby these elements are generated either by destroying the whole organism or the growth cultures of such microorganisms and subsequent purification steps yielding in the desired structure(s), or by synthetic processes induced by an appropriate manipulation of a suitable system such as, but not restricted to, bacteria, insects, mammalian, or other species, plus subsequent isolation and purification procedures or by induction of said synthetic processes in the animal needing a vaccine by direct incorporation of genetic material using suitable pharmaceutical compositions (polynucleotide vaccination). A vaccine may comprise one or simultaneously more than one of the elements described above.

The present vaccines may include a pharmaceutically acceptable carrier, excipient, carrier, stabilizer and/or diluent. Without intending to be limiting, examples include wetting agents and lubricating agents, preservative agents, lipids, stabilizers, solubilizers and emulsifiers. The carrier can be a solvent or dispersion medium containing, for example, water, ethanol, polyol (for example, glycerol, propylene glycol, liquid polyethylene glycol, and the like), suitable mixtures thereof and vegetable oils. One possible carrier is a physiological salt solution. Examples of stabilizers include, for example, glycerol/EDTA, carbohydrates (such as sorbitol, mannitol, trehalose, starch, sucrose, dextran or glucose), proteins (such as albumin or casein) and protein degradation products (e.g., partially hydrolyzed gelatin).

It is possible to provide an adjuvant in the vaccine. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses. Immunostimulatory agents or adjuvants have been used for many years to improve the host immune responses to, for example, vaccines. The vaccines of the present invention may be used in conjunction with adjuvants, for example, lipopolysaccharides, aluminum hydroxide and aluminum phosphate (alum), saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, and liposomes. Desirable characteristics of ideal adjuvants may include: (1) lack of toxicity; (2) ability to stimulate a long-lasting immune response; (3) simplicity of manufacture and stability in long-term storage; (4) ability to elicit both CMI and HIR to antigens administered by various routes; (5) synergy with other adjuvants; (6) capability of selectively interacting with populations of antigen presenting cells (APC); (7) ability to specifically elicit appropriate T-cell helper 1 (TH 1) or TH 2 cell-specific immune responses; and (8) ability to selectively increase appropriate antibody isotype levels (for example, IgA) against antigens. An adjuvant used with the present compositions and methods need not possess all these characteristics to be used.

As used herein, “immunogenically effective amount” refers to an amount, which is effective in reducing, eliminating, treating, preventing or controlling the symptoms of the infections, diseases, disorders, or condition.

The quantity to be administered depends on the subject to be treated, including, for example, the capacity of the immune system of the individual to mount a protective response. Suitable regimes for initial administration and booster doses are also variable but may include an initial administration followed by subsequent administrations. For example, it may be desirable to provide for an initial administration of the vaccine followed by additional doses. The need to provide an effective amount of the protective molecule will also need to be balanced with cost of providing higher amounts of the protective molecule. A cost effective vaccine is one in which the cost of producing it is less than the value one can obtain from using it. Measurement and determination of efficacy of any of the compositions and vaccines of the invention may be accomplished by any of the many methods available to one skilled in the art.

In one embodiment, a straightforward and quick method can be to perform a Western blot analysis of a sample candidate vaccine composition to quantitate the amount of polypeptide or fragment thereof in the sample. In one embodiment, one compares the amount of polypeptide to a standard known to be effective with like polypeptides from other biotypes, and either prepares a vaccine where the level of polypeptide produced is at least at this standard or higher or may test the vaccine with a test animal.

The compounds described herein can be administered to a subject at therapeutically effective doses to prevent PEDV-associated diseases. The dosage will depend upon the host receiving the vaccine as well as factors such as the size, weight, and age of the host.

The precise amount of immunogenic composition of the invention to be employed in a formulation will depend on the route of administration and the nature of the subject (e.g., age, size, stage/level of disease), and should be decided according to the judgment of the practitioner and each subject's circumstances according to standard clinical techniques. An effective immunizing amount is that amount sufficient to treat or prevent a PEDV infectious disease in a subject.

Immunogenicity of a composition can be determined by monitoring the immune response of test subjects following immunization with the composition by use of any immunoassay known in the art. Generation of a humoral (antibody) response and/or cell-mediated immunity may be taken as an indication of an immune response.

The immune response of the test subjects can be analyzed by various approaches such as: the reactivity of the resultant immune serum or mucosal secretions to the immunogenic conjugate, as assayed by known techniques, e.g., enzyme linked immunosorbent assay (ELISA), immunoblots, immunoprecipitations, virus neutralization, etc.; or, by protection of immunized hosts from infection by the pathogen and/or attenuation of symptoms due to infection by the pathogen in immunized hosts as determined by any method known in the art, for assaying the levels of an infectious disease agent, e.g., the viral levels (for example, by culturing of a sample from the subject), or other technique known in the art. The levels of the infectious disease agent may also be determined by measuring the levels of the antigen against which the immunoglobulin was directed. A decrease in the levels of the infectious disease agent or an amelioration of the symptoms of the infectious disease indicates that the composition is effective.

The therapeutics of the invention can be tested in vitro for the desired therapeutic or prophylactic activity, prior to in vivo use in animals. For example, in vitro assays that can be used to determine whether administration of a specific therapeutic is indicated include in vitro cell culture assays in which appropriate cells from a cell line or cells cultured from a subject having a particular disease or disorder are exposed to or otherwise administered a therapeutic, and the effect of the therapeutic on the cells is observed.

Alternatively, the therapeutics may be assayed by contacting the therapeutic to cells (either cultured from a subject or from a cultured cell line) that are susceptible to infection by the infectious disease agent but that are not infected with the infectious disease agent, exposing the cells to the infectious disease agent, and then determining whether the infection rate of cells contacted with the therapeutic was lower than the infection rate of cells not contacted with the therapeutic. Infection of cells with an infectious disease agent may be assayed by any method known in the art.

In addition, the therapeutics can be assessed by measuring the level of the molecule against which the antibody is directed in the animal model or human subject at suitable time intervals before, during, or after therapy. Any change or absence of change in the amount of the molecule can be identified and correlated with the effect of the treatment on the subject. The level of the molecule can be determined by any method known in the art.

After vaccination of an animal to PEDV using the methods and compositions of the present invention, any binding assay known in the art can be used to assess the binding between the resulting antibody and the particular molecule. These assays may also be performed to select antibodies that exhibit a higher affinity or specificity for the particular antigen. As one measure of vaccine potency, an ELISA can be performed on a sample collected from an individual vaccinated to determine whether antibodies to a vaccine comprising the sequence, a derivative, a homologue or a variant or fragment thereof generated anti-polypeptide antibodies. The individual's sample is measured against a reference anti-polypeptide antibody. Analysis of symptoms and measurement of animal weight gain also demonstrate lessening of impact of the disease in the presence of a particular dose. Fluorescent focused neutralization assay is still another assay to detect serum neutralizing antibodies and analyze effectiveness of a vaccine and a particular dose.

When testing animals administered the vaccine, for example, measuring antibody response is also effective in determining efficacy of the vaccine. Sera may be collected and titer measured as the reciprocal of the maximal dilution at which hemagglutination is inhibited, as described in an example below. Other measurements post-administration of the vaccine can also be employed to determine effectiveness, such as levels of anti-PEDV antibodies in mucosal secretions, pathological evaluation, isolation of the pathogen, measurement of symptoms, and overall health and weight gain of the animal.

Thus, the effectiveness of the present vaccine may also be evaluated quantitatively (for example, a decrease in the percentage of diseased tissue as compared to an appropriate control group) or qualitatively (e.g., isolation of virus from blood, detection of virus antigen in a tissue sample by an assay method, etc.). The symptoms of the disease may be evaluated quantitatively (e.g., temperature/fever), semi-quantitatively (e.g., severity of distress, or qualitatively (e.g., the presence or absence of one or more symptoms or a reduction in severity of one or more symptoms). Clearly one skilled in the art has many different options available for measuring effectiveness of the vaccine. With the present invention, it is possible to achieve protection against disease in a pig, and which protection is provided for longer periods than have been achieved in such animals. Protection periods of more than seven days after at least one challenge or exposure to the pathogenic microorganism have been achieved, and protection of at least two weeks, at least 20 days, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days or more, have been achieved using the invention. Such protection periods are also provided when using the invention with other animals. The protective response is also shown here in an embodiment to be specific to the disease as opposed to another disease, and thus demonstrates specific memory.

The vaccine can be administered via any convenient method including intranasal, oral and/or parenteral (e.g., intramuscular) administration. For example, the S1-LTB fusion protein containing vaccine can be administered intramuscularly one or more times. In another embodiment of the method, for example, the vaccine is administered orally one or more times. In an alternative embodiment oral administration can be followed by and/or precede administration of the vaccine at least once, intramuscularly. The maize grain can be fed to the animal, thereby reducing cost and loss of antigen that can occur through further processing.

The following is provided by way of illustration without intending to be limiting of the scope of the invention. All references cited herein are incorporated herein by reference.

### EXAMPLES

### Example 1

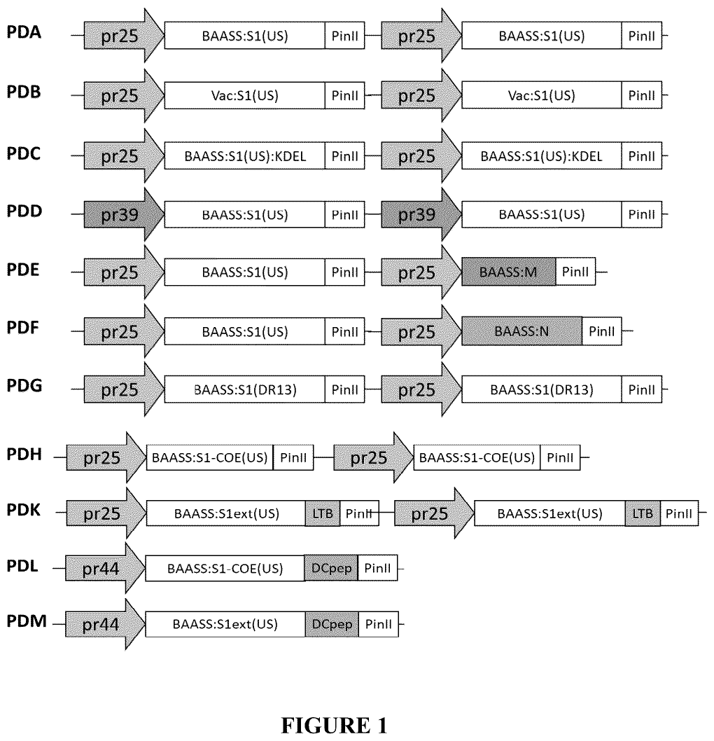

The Spike (S1) nucleotide sequence set forth below was introduced into constructs as outlined below and as shown in FIG. 1. S1 refers to the 2154 bp nucleotide sequence set forth in FIG. 1. S1(ext) refers to a 2307 bp sequence (SEQ ID NO: 1). The protein encoded by the sequences is SEQ ID NO: 2. BAASS refers to the barley alpha amylase signal sequence (SEQ ID NO: 3, the polypeptide encoded is SEQ ID NO: 4), PinII refers to the potato proteinase inhibitor polyadenylation sequence, M refers to a PEDV matrix protein encoding nucleotide sequence (SEQ ID NO: 7, the polypeptide encoded is SEQ ID NO: 8), N refers to PEDV N protein (a SEQ ID NO: 10, the polypeptide encoded is SEQ ID NO: 11); DR13 refers to a South Korea strain of the virus (SEQ ID NO: 25 is the nucleotide, the encoded polypeptide is SEQ ID NO: 9); COE refers to a small portion of the S1 protein that is involved in the immune response and the sequence set forth below (SEQ ID NO: 12). DCpep refers to the dendritic binding peptide. An embodiment provides the Spike polypeptide is fused to a dendritic cell targeting sequence, (DC3) (SEQ ID NO: 13), and/or a heat labile enterotoxin B subunit (LtB) peptide (SEQ ID NO: 14 and the polypeptide encoded is SEQ ID NO: 15). Dendritic cells are antigen-presenting cells that participate in activation of T cells. Polypeptides may be targeted to dendritic cells. See Mohamadzadeh et al. (2009) “Dendritic cell targeting of Bacillus anthracis protective antigen expressed by Lactobacillus acidophilus protects mice from lethal challenge” Proc. Natl. Acad. Sci USA 106, 4331-4336.

Reference to the promoter pr25 refers to the maize globulin-1 gene (SEQ ID NO: 16), pr39 refers to a maize 27 kD gamma-zein gene promoter (SEQ ID NO: 17); and pr44 refers to the pr25 globulin-1 promoter, with two extra copies of a portion of the promoter (SEQ ID NO: 18).

All fragments were optimized for maize codon usage and synthesized by Genescript. Full length coding sequence fragments were synthesized for the constructs with the US or DR13 strains with the BAASS signal sequence, as well as for the COE-DC peptide construct. Constructs with vacuolar or KDEL signal sequences (SEQ ID NO: 19) were prepared by synthesis of partial fragments and reconstruction of the full coding region using NcoI, EcoR1 and HindIlI restriction sites to exchange with the BAASS full length S1 synthesized fragments. A partial fragment was also ordered for the S1 Ext and used to reconstruct the full coding region by restriction digestion with HindIII+PacI. Cloning into the pSB11 vector was by NcoI and PacI restriction sites. Constructs with double copies of the complete promoter+coding region transcription unit were prepared by digestion with AscI and MluI and ligation of the second copy of the transcription unit.

The entire PEDV sequence is SEQ ID NO: 21, with the Spike protein encoded SEQ ID NO: 22.

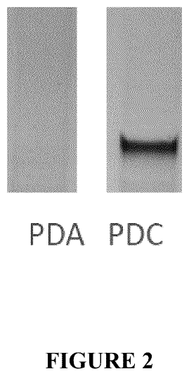

The S protein is extremely difficult to express in other hosts and therefore we did not know what to expect in the maize grain. It was surprising that the construct PDA with an apoplast targeting sequence provided poor expression levels of the protein while the PDC construct with an ER targeting sequence demonstrated good levels of expression (See FIG. 2).

### Example 2

### 1. Material

Seed from construct PDC (T1) was used for this trial. The T1 seed (800 g @13.5% moisture) was brought up to 21% moisture using USP sterile water and soaking overnight at ambient temperature in preparation of processing.

Some of the T1 seed was planted in the greenhouse to obtain enough seed for the trial (T2 seed). Six ears were selected at random to evaluate the presence of S1 antigen and the results showed that 5 out of the six ears were expressing as expected. 2100 g of T2 seed @11% moisture was therefore pooled (SG180001-SG180004) and soaked overnight as above to obtain a final moisture of 22%. Both the T1 and T2 seed were then combined to obtain 3,600 g of PDC seed.

The seed was put through a pin mill and then hand sieved through a #6 mesh screen to enrich for germ. The enriched germ fraction was dried to 11% and then put through the Glen Mill grinder to obtain corn meal. This was then sieved through a #10 mesh screen to remove pericarp. The final PDC meal was then distributed into 50 ml centrifuge tubes containing 14.5 g of meal/tube. This process was repeated using commercial corn seed.

For quantitation, ELISA and Westerns used a much smaller protein containing the COE domain as the standard as recombinant full length S1 protein is not available at this time. Based on the COE standard, estimates of S1 ranged from 0-40 mg/kg seed in T1 seed with most of the T1 seed and T2 seed testing at 3-10 mg/kg seed. With a moderate increase in concentration due to germ enrichment and a dosage of 29 grams/day, ELISA estimated indicate a dosage of ˜1 mg of S1/day.

TABLE 1
Treatment Groups:

| Group | Primary administration | Boost |
|---|---|---|
| A | Inject | Inject |
| B | Inject | Oral S1 maize |
| C | Inject | Oral Control maize |
| D | Oral S1 Maize | Oral S1 Maize |
| E | Oral Control Maize | Oral Control mazie |

### 2. Trial Initiation

Animals used in the study were 18-21 days old. Each treatment group consist of 4 animals. Animals were placed in 5 pens such and each pen will represent a separate treatment group. The treatment groups consist of that in Table 1. Material used for oral delivery with letter codes (B-E) that correspond to the treatments shown in Table 1.

### 3. Pre Challenge Activity

a. All animals will be observed daily for changes in general health. Daily observations will be recorded.
b. Daily environmental conditions will be recorded and will include room temp hi/low and a check of the feeders and waterers.
c. Fecal and serum samples to be taken and stored frozen on the days indicated in Table 2. When administration of the candidates and sample collection fall on the same day, all samples will be taken prior to administration of injected or oral material. Sera and fecal sample collection will be recorded
d. Blood will be collected (minimum of 5 ml) clotted and sera centrifuged and placed in vials.
e. One serum sample will be distributed into three vials (minimum, 1 ml each) and then frozen. The freezers temperatures will be monitored daily and the hi/low temperatures recorded manually.
f. Fecal swabs will be obtained. The fecal material will be divided into three vials and frozen (minimum 0.1 gram/vial).
g. When ready to ship, one frozen sample from the Tier 1 sample list in Diagnostic section below, will be shipped on dry ice to the diagnostic lab. The remainder kept frozen for sample retention.
h. Animals will be administered oral vaccine candidates according to the schedule in Table 2, below. Animals will be individually fed 2 labeled tubes/day (29 g material).
i. Animals will be given the parenteral vaccine according to the schedule in Table 2, below using the recommended dosage (Zoetis killed vaccine 2 ml IM).
j. Animals will be observed for any changes in health during the course of the study.
k. Weights will be taken of the animals just prior and to challenge and at the end of the study.

### 4. Challenge Activity

a. Virus for the challenge will be obtained (isolate PEDV USA/NC49469/2013).
b. PEDV will be given by IG for a full dose of (10 ml of 10\hat{ }4-10 \hat{ }5 TCID50/ml).
c. Fecal and serum samples will be collected, as above, according to the schedule in Table 2.
d. Daily observations will be done with special attention to diarrhea symptoms and weights taken every 4 days.

TABLE 2
Activity:
MZ-S-18-1221

| | | Day | | Gen | Clin | | | |
|---|---|---|---|---|---|---|---|---|
| DOT | Date | of Wk | Activity | obs | obs | Blood | Feces | Weigh |
| −6 | 7 Aug. 2018 | tue | arrival | x | | | | |
| −5 | 8 Aug. 2018 | wed | allot to treatments | x | | | | x |
| −4 | 9 Aug. 2018 | thu | | x | | | | |
| −3 | 10 Aug. 2018 | fri | | x | | | | |
| −2 | 11 Aug. 2018 | sat | | x | | | | |
| −1 | 12 Aug. 2018 | sun | | x | | | | |
| 0 | 13 Aug. 2018 | mon | Administer Trts | x | | x | x | x |
| | | | to Gps A-E | | | | | |
| 1 | 14 Aug. 2018 | tue | Administer Trts | x | | | | |
| | | | to Gp D, E | | | | | |
| 2 | 15 Aug. 2018 | wed | Administer Trts | x | | | | |
| | | | to Gp D, E | | | | | |
| 3 | 16 Aug. 2018 | thu | | x | | | | |
| 4 | 17 Aug. 2018 | fri | | x | | | | |
| 5 | 18 Aug. 2018 | sat | | x | | | | |
| 6 | 19 Aug. 2018 | sun | | x | | | | |
| 7 | 20 Aug. 2018 | mon | | x | | | | |
| 8 | 21 Aug. 2018 | tue | | x | | | | |
| 9 | 22 Aug. 2018 | wed | | x | | | | |
| 10 | 23 Aug. 2018 | thu | | x | | | | |
| 11 | 24 Aug. 2018 | fri | | x | | | | |
| 12 | 25 Aug. 2018 | sat | | x | | | | |
| 13 | 26 Aug. 2018 | sun | | x | | | | |
| 14 | 27 Aug. 2018 | mon | | x | | | | |
| 15 | 28 Aug. 2018 | tue | | x | | | | |
| 16 | 29 Aug. 2018 | wed | | x | | | | |
| 17 | 30 Aug. 2018 | thu | | x | | | | |
| 18 | 31 Aug. 2018 | fri | | x | | | | |
| 19 | 1 Sep. 2018 | sat | | x | | | | |
| 20 | 2 Sep. 2018 | sun | | x | | | | |
| 21 | 3 Sep. 2018 | mon | | x | | | | |
| 22 | 4 Sep. 2018 | tue | | x | | | | |
| 23 | 5 Sep. 2018 | wed | | x | | | | |
| 24 | 6 Sep. 2018 | thu | | x | | | | |
| 25 | 7 Sep. 2018 | fri | | x | | | | |
| 26 | 8 Sep. 2018 | sat | | x | | | | |
| 27 | 9 Sep. 2018 | sun | | x | | | | |
| 28 | 10 Sep. 2018 | mon | Administer Trts | x | | x | x | x |
| | | | to Gps A-E | | | | | |
| 29 | 11 Sep. 2018 | tue | Administer Trts | x | | | | |
| | | | to Gp B-E | | | | | |
| 30 | 12 Sep. 2018 | wed | Administer Trts | x | | | | |
| | | | to Gp B-E | | | | | |
| 31 | 13 Sep. 2018 | thu | | x | | | | |
| 32 | 14 Sep. 2018 | fri | | x | | | x | |
| 33 | 15 Sep. 2018 | sat | | x | | | | |
| 34 | 16 Sep. 2018 | sun | | x | | | | |
| 35 | 17 Sep. 2018 | mon | | x | | | x | |
| 36 | 18 Sep. 2018 | tue | | x | | | | |
| 37 | 19 Sep. 2018 | wed | | x | | | | |
| 38 | 20 Sep. 2018 | thu | | x | | | x | |
| 39 | 21 Sep. 2018 | fri | | x | | | | |
| 40 | 22 Sep. 2018 | sat | | x | | | | |
| 41 | 23 Sep. 2018 | sun | | x | x | | | |
| 42 | 24 Sep. 2018 | mon | Challenge all gps | x | x | x | x | x |
| 43 | 25 Sep. 2018 | tue | | x | x | | | |
| 44 | 26 Sep. 2018 | wed | | x | x | | x | |
| 45 | 27 Sep. 2018 | thu | | x | x | | | |
| 46 | 28 Sep. 2018 | fri | | x | x | | x | x |
| 47 | 29 Sep. 2018 | sat | | x | x | | | |
| 48 | 30 Sep. 2018 | sun | | x | x | | x | |
| 49 | 1 Oct. 2018 | mon | | x | x | | | |
| 50 | 2 Oct. 2018 | tue | | x | x | | x | x |
| 51 | 3 Oct. 2018 | wed | | x | x | | | |
| 52 | 4 Oct. 2018 | thu | | x | x | | x | |
| 53 | 5 Oct. 2018 | fri | | x | x | | | |
| 54 | 6 Oct. 2018 | sat | | x | x | | x | x |
| 55 | 7 Oct. 2018 | sun | | x | x | | | |
| 56 | 8 Oct. 2018 | mon | | x | x | | | |
| 57 | 9 Oct. 2018 | tue | Euthanize pigs | x | x | x | x | x |

### 5. Diagnostics

a. Tier 1
   i. Neutralization assays will be performed 1 for sera from day 42 and 57 of all pigs
   ii. Neutralizing activity from fecal samples of day 35, 42 of all pigs
   iii. PCR will be used to detect virus shedding in feces at day 42, 46, 50, 54, and 57 for pigs from in all groups.
b. Tier 2
   i. If neutralizing activity is detected in sera or fecal matter then additional tests may be requested for days not initially tested.
   ii. Additional PCR tests may be done depending on the initial test for days not tested.
   iii. Any test may be repeated for any reason.
c. Titers for anti-PEDV sera antibodies (IgA and IgG) or IgA in fecal samples will be done at the conclusion of the study.

### Example 3

In this work, we evaluated constructs containing the S1 subunit of the spike protein in transgenic maize targeted to various cellular locations as well as S1 fused to carrier proteins with the goal of accumulating the S1 antigen at concentrations high enough to use the maize grain directly for oral administration of the vaccine candidate. The highest levels were obtained in constructs targeted to the endoplasmic reticulum (ER) or as fusions with the B subunit of E. coli heat-labile enterotoxin or a dendritic cell binding peptide. Maize material harboring the construct targeting S1 to the ER was then orally administered to pigs and shown to elicit neutralizing antibodies. This opens the possibility of using maize-produced antigen as a practical approach for a new type of PEDV vaccine.

We have produced maize lines targeting expression of PEDV spike protein to various subcellular locations and in combination with proteins known to increase immunogenicity. Relatively high accumulation of the S1 portion of the spike protein was observed using a construct targeted to the endoplasmic reticulum (ER) as well as using fusions of S1 with the E. coli heat labile subunit (LTB) and a dendritic cell (DC) binding peptide that may act as carriers to increase immunogenicity. Expression levels were at least 5 mg/kg of whole seed. S1 ER-targeted maize material was orally administered to piglets and elicited a serum neutralizing antibody response. To our knowledge, this is the first report of an immune response in pigs using plant-produced material expressing PEDV S1 to elicit neutralizing antibodies on oral delivery. This proof-of-concept provides encouragement for this approach in the development of an effective oral vaccine against this disease.

### Methods

### Preparation of Constructs

The sequence of the spike protein from the PEDV strain Colorado 2013 (Genbank KF272920) was optimized for maize codon usage (See Examples above). The nucleotide sequence of the coding region was outsourced for commercial gene synthesis by GenScript. Six different constructs were prepared (FIG. 1) with varying subcellular localizations and fusions. The native signal sequence was replaced with a barley alpha amylase signal sequence for cell wall targeting in constructs PDA, PDC, PDD, PDK, and PDM and with a vacuolar targeting sequence in construct PDB. The S1 coding region (aa 23-738 for constructs PDA, PDB, PDC, and PDB or aa 23-789 for constructs PDK and PDM) was synthesized for transfer into the maize transformation vector pSB11¹⁸ using NcoI and PacI restriction sites. For the other constructs, smaller portions of the coding region containing, for example, the KDEL ER targeting sequence, were commercially synthesized to exchange with portions of the full-length coding region by restriction digestion and ligation. All constructs except one, PDD, incorporated the S1 coding region under control of a promoter derived from the maize globulin-1 gene (pr25 or pr44) which target expression to the embryo. In construct PDM an engineered version of the pr25 promoter, pr44, contains two extra copies of the 5′ region of pr25¹⁹. One construct (PDD) incorporated the S1 protein under control of a promoter (pr39, derived from the maize 27 kDa gamma zein gene) targeting expression to the endosperm. Each transcription unit also incorporated the terminator from potato proteinase inhibitor II. Subcellular localization and terminator sequences were as previously described¹⁹.

### Maize Transformation

Maize transformation was carried out as previously described with modifications²⁰. In brief, the constructs were transferred into the LBA4404 Agrobacterium strain containing the vector pSB1 by a triparental mating procedure¹⁸. The cointegrate DNA was then electroporated into Agrobacterium tumefaciens strain EHA101²¹. Hill maize embryos roughly 1.5 to 3 mm in length were mixed with A. tumefaciens EHA101 with the appropriate vector for transformation²⁰. Plants from events selected on bialaphos were grown to maturity in the greenhouse and pollinated with Hill to produce T1 generation seed.

### Western Blot Analysis

A small antigenic portion of the S1 protein known as the core neutralizing epitope (COE, aa 494-641) was cloned, expressed and purified by GenScript. The resulting protein was used as an immunogen to prepare polyclonal anti-S1 antibodies in rabbit by Pacific Immunology. Ground maize seed was extracted in 1×PBS+1% SDS, loaded onto a 4-12% bis-tris gel (LifeTech), and transferred to PDVF membrane by iBlot. The blot was incubated in Pacific Immunology's custom rabbit anti-PEDV S1 overnight and developed with anti-rabbit-alkaline phosphatase conjugate (Jackson Immunoresearch #111-055-003)) and BCIP-NBT liquid substrate (Sigma). The concentration of S1 was estimated using the COE standard synthesized by Genscript. Levels were at least 10 mg/kg of whole seed.

### Pig Study

### Preparation of Material

Several high-expressing maize lines were identified for construct PDC. Seed from the first generation of plants (T1) was planted to obtain additional grain (T2) for pig studies. T1 and T2 generation material was pooled and used for the animal studies. Since expression was targeted to the embryo, the antigen was further concentrated for S1 by enriching for the germ fraction using customized small scale processing equipment. The germ fraction was then dried to obtain a moisture content of 11% and ground such that >80% of the material could pass through a 20 mesh screen.

### Animals and Treatment Groups

A double-blind study was conducted by Veterinary Resources Inc. (VRI, Ames, IA). Twelve pigs 18-21 days old were used (Wilson's Prairie View Farms, Burlington, WI) and determined to be free from PEDV by PCR analysis of fecal material and by serum neutralizing tests performed by South Dakota State University Diagnostics Laboratory. The pigs were randomly divided on a weight basis into treatment groups and housed in separate pens. Pigs were fed Ultra Care 240 or 500 Med. A primary dose was administered after six days acclimation. PDC maize material was fed to pigs as a prime and booster dose (Table 3). The boost occurred two weeks after the primary dose. Another treatment consisted of pigs that were injected with a commercial virus-based vaccine, while a third group was administered untransformed ground maize germ. Animals (4 per treatment) were either fed recombinant or control ground maize material (29 g/day on three successive days for a total of 87 g) or injected intramuscularly with a commercially available killed virus vaccine (Zoetis). The animals were fasted for four hours before being offered the maize material and returned to their normal diet an hour after administration. Each animal was hand fed and consumed the full dose of maize material offered. At day 28 a booster dose of oral material or injection was administered.

### Challenge, Sample Collection, and Analysis of Serum Response

Virus (isolate PEDV USA/NC49469/2013) for challenge was obtained from Dr. Jianqiang Zhang at Iowa State University. Challenge by intragastric inoculation was on day 42 after primary immunization with 10 mL at 10⁴ to 10⁵ TCID50/mL. Serum samples were collected on days 0 (pre-screen), day 42 (pre-challenge), and 15 days after challenge. Serum neutralizing antibody (SNA) response was determined by fluorescent focus neutralization assay at the South Dakota State University Diagnostics Laboratory. Fecal samples were collected and an assay for mucosal response will confirm such a response by the animal.

### Statistics

A mixed effects analysis of variance was used to analyze the data with the final titer as response variable of interest and the treatment group and the weight group as factors. Weight group was included in the analysis as a random effect to account for any variation in the titers due to animal weight. Differences in the treatment groups were compared using Tukey's HSD procedure with a 5% significance level. The data were analyzed on a base-2 log scale. Comparing the titers on the log scale detects significant differences between the treatment groups (p-value=0.05). The differences between groups were determined using the Tukey HSD method.

### Results

### Expression of S1.

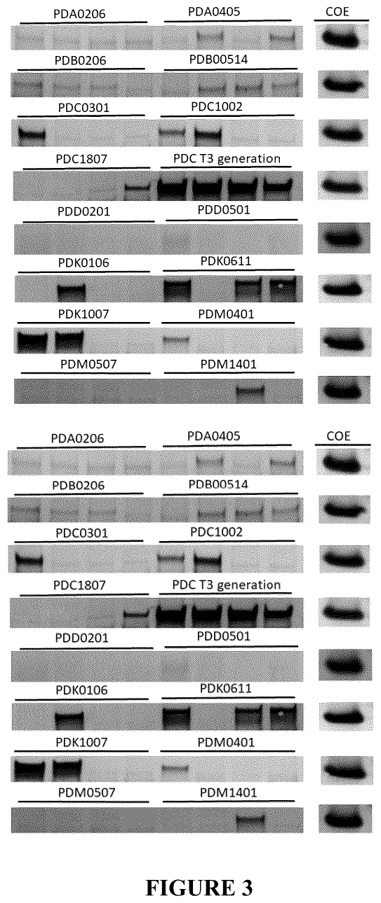

Constructs coding for the PEDV S protein S1 subunit labeled PDA, PDB, PDC, PDD, PDK AND PDM were prepared and transformed into maize to produce the subunit vaccine. See FIG. 1. Three constructs, PDC, PDK, and PDM, showed the highest levels of expression of PEDV S1 (>20 ug/g seed), based on Western blot analysis of single seeds (FIG. 3). Expression levels were at least 10 mg/kg of whole seed. Construct PDC codes for a portion of the spike protein S1 region incorporating amino acids 23-738 with a barley-alpha amylase signal sequence at the N-terminus and a KDEL sequence for endoplasmic reticulum (ER) retention at the C-terminus. Construct PDK contains an extended S1 protein incorporating amino acids 23-789, with the barley-alpha amylase signal sequence replacing the signal sequence at the N-terminus and fused to the E. coli heat-labile enterotoxin (LTB) at the carboxy terminus. Construct PDM contains the extended S1 protein fused to a dendritic cell targeting (DC) peptide at the carboxy terminus. In each case expression is under control of a promoter derived from the maize globulin 1 gene that directs expression to the embryo portion of the maize kernel, with the exception of construct PDD, in which the coding region is under control of an endosperm-targeted promoter. Also, in each of these cases the plant transcription unit was duplicated in the final construct. Constructs targeting expression to the cell wall (PDA), to the vacuole (PDB), or under control of a promoter targeting expression to the endosperm portion of the kernel (PDD) showed minimal expression relative to PDC, PDK, and PDM. Four representative individual seeds for the indicated plant were pulverized and extracted in 1×PBS+1% SDS. The protein was transferred to PVDF membrane and incubated with polyclonal rabbit antibody raised to commercially synthesized COE portion of the spike protein (shown as a positive control at the right). Note that in all cases except PDC T3 generation, T1 generation seed crossed to untransformed maize is shown and half of the seed are expected to be positive. PDC T3 has been self-crossed leading to expression in all seeds.

### Animal Study.

PDC maize material was prepared as ground meal and fed to pigs as a prime and booster dose (Table 3). Quantitation of S1 was estimated by comparison of the intensity of bands on western blots using the COE peptide as the standard. Based on this estimate, each dose contained approximately 0.8 mg of S1.

TABLE 3
Pig study design:

| Group | Prime | Boost |
|---|---|---|
| 1 | Injected | Injected |
| 2 | Oral S1 Maize | Oral S1 Maize |
| 3 | Oral Control Maize | Oral Control Maize |

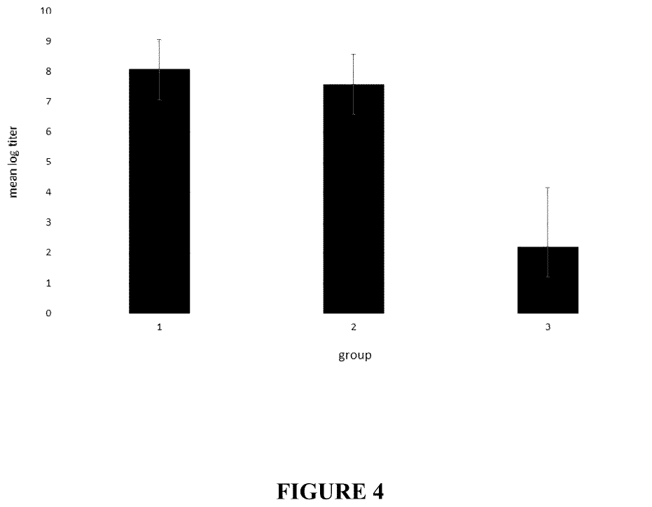

The pigs were screened before immunization and found to have a serum neutralizing antibody titer not above background. Serum neutralizing antibodies (SNA) were only observed prior to the challenge in pigs administered the injected vaccine. After the challenge, the pigs administered orally delivered antigens and injected vaccines both showed a significant increase in SNA over the control group. In both cases the response was statistically different from the control group administered untransformed maize. There was not, however, a statistically significant difference between the two vaccinated groups. Symptoms were monitored but only minor diarrhea was observed. This is most likely due to the age of the pigs (˜2 months), as newborn pigs are most susceptible to PEDV. Sera antibody titers were surprisingly high with the orally administered vaccine. In the control, viral neutralizing antibody titers observed were 10 in control animals, 200 in animals receiving oral vaccine and 340 in animals receiving injection vaccine. FIG. 4 shows Pig antibody response. FIG. 4 shows the mean of the log titer. Serum neutralizing antibody response from the indicated groups (n=4) were assessed by fluorescent focus neutralization assay at 15 days post-challenge (dpc), as described in the methods section. At 15 dpc treatment groups containing S1 (one and two) showed significant differences with the control group (three) with p-values for each comparison substantially below 0.05. There was no significant difference in the titers between treatment groups one (injected S1) and two (orally-delivered maize S1). Group 1 is the group receiving injection, Group 2 is the group receiving oral administration of plant material with the vaccine and Group 3 is the control group receiving oral administration of corn not comprising the transgene.

### Discussion

We have demonstrated relatively high levels of accumulation in maize grain of the PEDV spike protein S1, when targeted to the ER. This can allow for the cost efficient production of a subunit vaccine as this antigen has been very difficult to express in other systems. More critically, however, we have also shown that after the virus challenge, maize-produced orally delivered S1 was able to elicit serum neutralizing antibodies in pigs similar to those elicited by the commercial injected vaccine post challenge. Prior to the virus challenge, little serum neutralizing antibody response was detected in the group fed maize-produced S1 or controls. This observation is similar to earlier results with TGEV that showed significant titers only after the viral challenge. Presumably, this is due to a response below the limits of detection after the first boost of the vaccine. The relatively high serum response is surprising in that the response seen in the sera is normally low compared to the response in mucosal tissues when candidates are orally delivered. This is encouraging as the mucosal response is likely to be even more robust and may be more protective for this disease than the serum response. Future work will include development of reliable methods for detection of mucosal response.

Newborn piglets are most susceptible to PEDV and clinical symptoms in this study were minimal after the viral challenge in all groups due to the older age of the pigs. The ideal commercial vaccine will use vaccination of sows to allow for lactogenic immunity from colostrum to be passed to suckling piglets. Additional material is being generated that will allow more extensive studies addressing lactogenic immunity. This may require higher levels of antigen for the much larger dams. If needed, this can be achieved either by increasing the S1 concentrations using selection, as described earlier for other antigens²² and shown in FIG. 2 (PDC T3 generation), for PEDV S1 or by feeding larger amounts of maize flour to the sows. The maize system has many inherent properties making it amenable to development of practical low-cost oral vaccines for livestock. In addition to high expression levels, a plant-produced antigen should be stable to allow for a low cost of production and storage. Seeds such as maize grain have evolved to maintain proteins in a stable environment, allowing germination after years of dormancy, in contrast to vegetative tissue or fruits that undergo degradation shortly after harvest. This stability has been demonstrated for recombinant proteins that retain activity after being stored for years in the grain and allows for long-term storage, transport at ambient temperatures, and processing of the grain at will rather than a requirement to process large batches immediately upon harvest¹⁹. Stability can also be further increased by removal of oils by supercritical fluid extraction (SFE), a process we have demonstrated for other antigens²³. Bioencapsulation of the antigen in maize grain has been shown to help maintain antigenic determinants during passage through the digestive system leading to a higher immune response relative to purified protein, and the immune response may also be enhanced upon SFE treatment as has been shown in other cases²³.

In conclusion, we have demonstrated high levels of expression of a recalcitrant antigen, PEDV spike protein, in transgenic maize. After optimization, this system should result in a heat-stable oral vaccine for PEDV that does not require a cold chain. Once the system is established for one PEDV strain it should be possible to develop maize lines to incorporate antigens for additional strains as needed.

FIG. 4 shows Pig antibody response. Serum neutralizing antibody response from the indicated groups (n=4) were assessed by fluorescent focus neutralization assay at 15 days post-challenge (dpc), as described in the methods section. At 15 dpc treatment groups containing S1 (one and two) showed significant differences with the control group (three) with p-values for each comparison substantially below 0.05. There was no significant difference in the titers between treatment groups one (injected S1) and two (orally-delivered maize S1). Groups received either two injected or two oral doses.

### Example 4

In this study, the production of different molecular weight S1 protein entities was analyzed using gel chromatography to determine if the PDC or PDK constructs might produce higher molecular weight S1 proteins, which might indicate the formation of multimers.

### Methods

### PDK DNA Construct Generation

The S1 coding region was codon optimized and synthesized such that it could be utilized for transfer into the corn transformation vector pSB11 (using an NcoI overlapping the initiating ATG and a PacI restriction site in the terminator region to add the coding region downstream of the promoter). The native signal sequence was replaced with a barley alpha amylase signal sequence (BAASS) for cell wall targeting. The construct incorporated the S1 coding region under control of a promoter derived from the maize globulin-1 gene (pr25) which targets expression to the embryo. Each transcription unit also incorporated the terminator from potato proteinase inhibitor II. The construct had two copies of the entire transcription unit incorporating the promoter and the S1 coding sequence. In addition to the open reading frame for S, a second version of S was created by fusing the carrier protein, LTB (the non-toxic subunit of the heat-labile toxin from E. coli) to S. Both constructs, PDC (S1 protein) and PDK (S1:LTB protein), were then used to transform maize.

### Transformation of Maize

DNA was transformed (by electroporation) into Agrobacterium tumefaciens strain and inoculated in a small (2 ml) overnight culture and transformed bacteria were selected. Corn embryos were dissected and mixed with A. tumefaciens (in log phase growth). Embryos were placed on solid media and incubated for 4 days; next they were transferred to media with bialaphos for selection. Transformation events which grow in the selection media are the ones that were successfully transformed with A. tumefaciens. Embryos were grown to regenerate plants, next they were transferred to the greenhouse and grown to maturity (when mature they were pollinated with untransformed corn to produce T1 generation seed).

### S Protein Expression Analysis

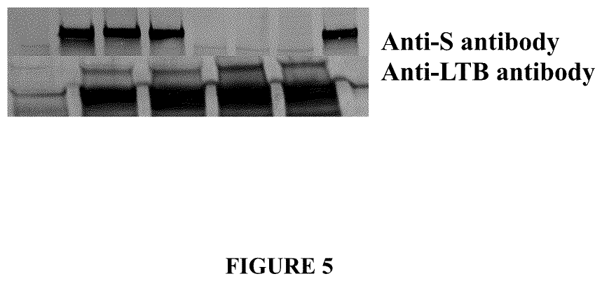

Transformed corn samples were grounded, and protein was isolated using PBS. Western blotting analysis confirmed the expression of S protein with anti-S and anti-LTB antibodies. The dark bands in FIG. 5 represent a protein of ˜180 KD and the light bands are extracts from seeds that do not contain the expression vector.

### Sample Preparation

Maize grain engineered to express the spike protein (S) from Porcine Epidemic Diarrhea virus (PDC construct), or the S protein fused with E. coli heat labile subunit (LTB) (PDK construct) was ground to obtain flour. The samples were extracted using 2 g of ground maize in 10 mL of extraction buffer (PBS). The extract was centrifuged at 2,000×g for 15 min at 2-8° C. and the supernatant was filtered through Whatman 1.

### Gel Chromatography

Filtered sample supernatant was loaded onto a calibrated size exclusion chromatography column (Superdex 200, Cytiva) with a fractionation range (Mᵣ) of ˜10,000 to 600,000 to separate molecules by size. The dead volume was determined to be 3 mL and, subsequently, 0.5 mL fractions were collected from the column and stored at 2-8° C. for analysis.

### PEDV S Detection by ELISA

Column fractions were analyzed for the S protein using a sandwich ELISA. The S concentration of each fraction was graphed to visualize peaks of different molecular weight entities.

### Results and Conclusions

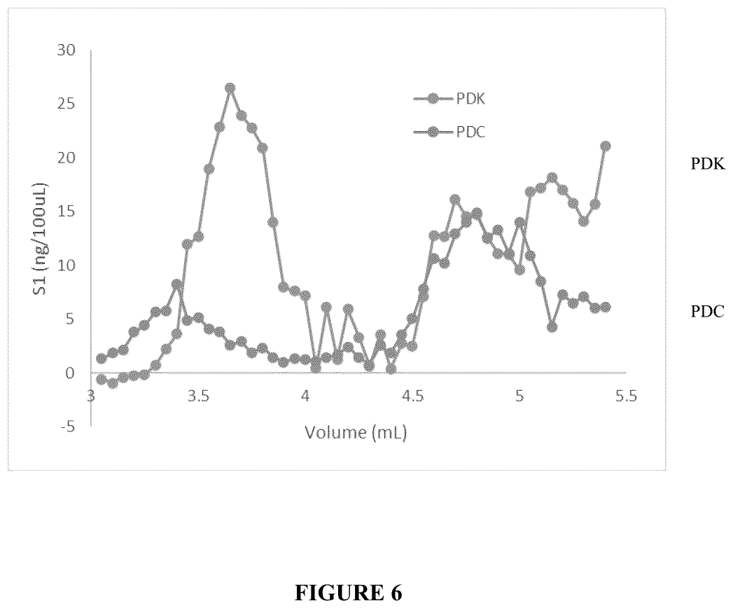

Results are shown in FIG. 6. The PDK construct showed a significantly higher ratio of high molecular weight S protein to low molecular weight S protein when compared to the PDC construct.

### Example 5

An animal trial was conducted to evaluate whether corn-expressing S1 protein with or without the LTB carrier protein can elicit a mucosal immune response.

Treatment groups consisted of 4 pigs each of 3 weeks old piglets:
1) injected commercial vaccine (INJ)
2) control corn (CON)
3) oral PDC vaccine (PDC)
4) oral PDK vaccine (PDK)

Animals received a dose by feeding the animals maize grain (200 g) containing the antigen (PDC=0.9 mg, PDK=0.3 mg/kg) on three consecutive days of the study (days 1-3), then boosted at 3 weeks of age (days 21-23), and a second booster dose on days 35-37. Fecal samples were collected multiple times during the study and stored in the freezer until further analysis.

### Fecal ELISA Assay

An ELISA assay was developed to measure the level of IgAs in porcine fecal samples.

An ELISA plate was coated with purified recombinant PEDV S1 protein in a carbonate/bicarbonate buffer and incubated overnight. The plate was blocked and subsequently incubated with extracted fecal samples. The plate was then incubated with HRP-conjugated goat anti-pig IgA. The substrate was added, the reaction was stopped, and absorbance was read.

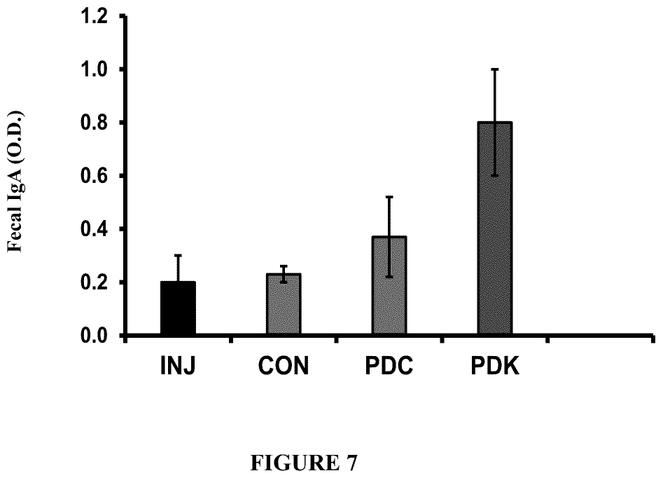

Results are shown in FIG. 7, which show that the PDK construct (with the PEDV S1 protein fused to the heat labile enterotoxin B subunit LTB) elicited a greater IgA response in animals than the PDC construct (with the PEDV S1 protein not fused to LTB).

### REFERENCES

[1] Gerdts, V., and Zakhartchouk, A. (2017) Vaccines for porcine epidemic diarrhea virus and other swine coronaviruses, Veterinary microbiology 206, 45-51.
[2] Guo, J., Fang, L., Ye, X., Chen, J., Xu, S., Zhu, X., Miao, Y., Wang, D., and Xiao, S. (2019) Evolutionary and genotypic analyses of global porcine epidemic diarrhea virus strains, Transboundary and emerging diseases 66, 111-118.
[3] Sun, D., Feng, L., Shi, H., Chen, J., Cui, X., Chen, H., Liu, S., Tong, Y., Wang, Y., and Tong, G. (2008) Identification of two novel B cell epitopes on porcine epidemic diarrhea virus spike protein, Veterinary microbiology 131, 73-81.
[4] Chang, S.-H., Bae, J.-L., Kang, T.-J., Kim, J., Chung, G.-H., Lim, C.-W., Laude, H., Yang, M.-S., and Jang, Y.-S. (2002) Identification of the epitope region capable of inducing neutralizing antibodies against the porcine epidemic diarrhea virus, Molecules and cells 14, 295-299.
[5] Makadiya, N., Brownlie, R., van den Hurk, J., Berube, N., Allan, B., Gerdts, V., and Zakhartchouk, A. (2016) S1 domain of the porcine epidemic diarrhea virus spike protein as a vaccine antigen, Virology journal 13, 1.
[6] Oh, J., Lee, K.-W., Choi, H.-W., and Lee, C. (2014) Immunogenicity and protective efficacy of recombinant S1 domain of the porcine epidemic diarrhea virus spike protein, Archives of virology 159, 2977-2987.
[7] Van Noi, N., and Chung, Y.-C. (2017) Optimization of expression and purification of recombinant S1 domain of the porcine epidemic diarrhea virus spike (PEDV-S1) protein in Escherichia coli, Biotechnology & Biotechnological Equipment 31, 619-629.
[8] Piao, D.-C., Lee, Y.-S., Bok, J.-D., Cho, C.-S., Hong, Z.-S., Kang, S.-K., and Choi, Y.-J. (2016) Production of soluble truncated spike protein of porcine epidemic diarrhea virus from inclusion bodies of Escherichia coli through refolding, Protein expression and purification 126, 77-83.
[9] Di-qiu, L., Jun-wei, G., Xin-yuan, Q., Yan-ping, J., Song-mei, L., and Yi-jing, L. (2012) High-level mucosal and systemic immune responses induced by oral administration with Lactobacillus-expressed porcine epidemic diarrhea virus (PEDV) S1 region combined with Lactobacillus-expressed N protein, Applied microbiology and biotechnology 93, 2437-2446.
[10] Hou, X., Jiang, X., Jiang, Y., Tang, L., Xu, Y., Qiao, X., Liu, M., Cui, W., Ma, G., and Li, Y. (2018) Oral Immunization against PEDV with Recombinant Lactobacillus casei Expressing Dendritic Cell-Targeting Peptide Fusing COE Protein of PEDV in Piglets, Viruses 10, 106.
[11] Bae, J.-L., Lee, J.-G., Kang, T.-J., Jang, H.-S., Jang, Y.-S., and Yang, M.-S. (2003) Induction of antigen-specific systemic and mucosal immune responses by feeding animals transgenic plants expressing the antigen, Vaccine 21, 4052-4058.
[12] Huy, N.-X., Kim, S.-H., Yang, M.-S., and Kim, T.-G. (2012) Immunogenicity of a neutralizing epitope from porcine epidemic diarrhea virus: M cell targeting ligand fusion protein expressed in transgenic rice calli, Plant cell reports 31, 1933-1942.
[13] Huy, N.-X., and Kim, M.-Y. (2019) Improved expression of porcine epidemic diarrhea antigen by fusion with cholera toxin B subunit and chloroplast transformation in Nicotiana tabacum, Plant Cell, Tissue and Organ Culture (PCTOC), 1-11.
[14] Lamphear, B. J., Jilka, J. M., Kesl, L., Welter, M., Howard, J. A., and Streatfield, S. J. (2004) A corn-based delivery system for animal vaccines: an oral transmissible gastroenteritis virus vaccine boosts lactogenic immunity in swine, Vaccine 22, 2420-2424.
[15] Hayden, C. A., Fischer, M. E., Andrews, B. L., Chilton, H. C., Turner, D. D., Walker, J. H., Tizard, I. R., and Howard, J. A. (2015) Oral delivery of wafers made from HBsAg-expressing maize germ induces long-term immunological systemic and mucosal responses, Vaccine 33, 2881-2886.
[16] Streatfield, S. J., Mayor, J. M., Barker, D. K., Brooks, C., Lamphear, B. J., Woodard, S. L., Beifuss, K. K., Vicuna, D. V., Massey, L. A., and Horn, M. E. (2002) Development of an edible subunit vaccine in corn against enterotoxigenic strains of Escherichia coli, In Vitro Cellular & Developmental Biology-Plant 38, 11-17.
[17] Man, K., Yang, B., Xue, J., Qin, X., Zhou, H., Xu, F., Xu, C., Yang, F., Zhang, L., and Li, X. (2014) Expression of core neutralizing epitope gene of porcine epidemic diarrhea virus in maize, Journal of Agricultural Science and Technology (Beijing) 16, 28-35.
[18] Komari, T., Hiei, Y., Saito, Y., Murai, N., and Kumashiro, T. (1996) Vectors carrying two separate T-DNAs for co-transformation of higher plants mediated by Agrobacterium tumefaciens and segregation of transformants free from selection markers, Plant Journal 10, 165-174.
[19] Hayden, C. A., Egelkrout, E. M., Moscoso, A. M., Enrique, C., Keener, T. K., Jimenez-Flores, R., Wong, J. C., and Howard, J. A. (2012) Production of highly concentrated, heat-stable hepatitis B surface antigen in maize, Plant Biotechnology Journal 10, 979-984.
[20] Ishida, Y., Saito, H., Ohta, S., Hiei, Y., Komari, T., and Kumashiro, T. (1996) High efficiency transformation of maize (Zea mays L.) mediated by Agrobacterium tumefaciens, Nat Biotechnol 14, 745-750.
[21] Hood, E. E., Helmer, G. L., Fraley, R. T., and Chilton, M. D. (1986) The hypervirulence of Agrobacterium tumefaciens A281 is encoded in a region of pTiBo542 outside of T-DNA, J Bacteriol 168, 1291-1301.
[22] Hood, E. E., Devaiah, S. P., Fake, G., Egelkrout, E., Teoh, K., Requesens, D. V., Hayden, C., Hood, K. R., Pappu, K. M., Carroll, J., and Howard, J. A. (2012) Manipulating corn germplasm to increase recombinant protein accumulation, Plant Biotechnology Journal 10, 20-30.
[23] Hayden, C. A., Smith, E. M., Turner, D. D., Keener, T. K., Wong, J. C., Walker, J. H., Tizard, I. R., Jimenez-Flores, R., and Howard, J. A. (2014) Supercritical fluid extraction provides an enhancement to the immune response for orally-delivered hepatitis B surface antigen, Vaccine 32, 1240-1246.

### LIST OF SEQUENCES

SEQ ID NO: 1 PEDV S1(US) nucleotide sequence
SEQ ID NO: 2 PEDV S1 ext nucleotide sequence
SEQ ID NO: 3 PEDV S1 (US) encoded peptide
SEQ ID NO: 4 PEDV S1(ext) encoded peptide
SEQ ID NO: 5 BAASS nucleotide sequence
SEQ ID NO: 6 BAASS encoded peptide
SEQ ID NO: 7 ABI Optimized PEDV-M (US)
SEQ ID NO: 8 Amino acid sequence PEDV M (US) encoded peptide
SEQ ID NO: 9 PEDV S1 DR13 (South Korean Strain) encoded polypeptide
SEQ ID NO: 10 ABI Optimized PEDV-N (US)
SEQ ID NO: 11 Amino acid sequence PEDV-N encoded peptide
SEQ ID NO: 12 PEDV COE nucleotide sequence
SEQ ID NO: 13 DC3 nucleotide sequence
SEQ ID NO: 14 LTB heat labile peptide nucleotide sequence
SEQ ID NO: 15 LTB heat labile encoded peptide
SEQ ID NO: 16 Promoter pr25—globulin 1
SEQ ID NO: 17 Promoter pr39—27 kDa gamma zein
SEQ ID NO: 18 Promoter P44 sequence
SEQ ID NO: 19 KDEL ER targeting sequence (nucleotide sequence)
SEQ ID NO: 20 PEDV nucleotide sequence encoding entire viral protein, (GenBank Accession No. KF272920)
SEQ ID NO: 21 Spike protein sequence encoded by SEQ ID NO: 16 (above) (See GenBank AGO58924.1)
SEQ ID NO: 22 Spike protein of Korean PEDV strain of Gen Bank AAM19716
SEQ ID NO: 23 Spike protein of China PEDV strain of GenBank AFL02627.1
SEQ ID NO: 24 HDEL amino acid sequence
SEQ ID NO: 25 DR13 nucleotide sequence

1. A method of producing a protective response to Porcine Epidemic Diarrhea Virus (PEDV) in an animal, the method comprising,
a) orally administering to said animal a composition comprising plant or plant product comprising a S1-LTB fusion protein, wherein said S1-LTB fusion protein comprises the Spike (S1) protein of PEDV operably fused to the heat labile enterotoxin B subunit (LTB) peptide, wherein said S1 protein comprises SEQ ID NO: 3, 4, 9, 21 or 22 or a sequence having at least 90% or at least 95% identity to SEQ ID NO: 3, 4, 9, 21 or 22 or a functional fragment of said S1 protein; and wherein said LTB peptide comprises SEQ ID NO: 15 or a sequence having at least 90% or at least 95% identity to SEQ ID NO: 15 or a functional fragment of said LTB peptide; and
b) producing a protective response to said PEDV in said animal.

2. The method of claim 1, wherein said protective response comprises a serum antibody response in said animal.

3. The method of claim 2, wherein said serum antibody response is at least 20 times greater than serum antibody response in an animal not administered said vaccine.

4. The method of claim 1, wherein said composition comprising said S1-LTB fusion protein elicits a greater mucosal immune response than a composition comprising said S1 protein without said LTB peptide operably fused thereto.

5. The method of claim 4, wherein said greater mucosal immune response comprises greater levels of fecal anti-PEDV immunoglobulin A (IgA) in said animal.

6. The method of claim 1, wherein said composition comprising said S1-LTB fusion protein comprises a higher ratio of high molecular weight S1 protein to low molecular weight S1 protein in comparison to a composition comprising said S1 protein without said LTB peptide operably fused thereto.

7. A method of producing a greater mucosal immune response to Porcine Epidemic Diarrhea Virus (PEDV) in an animal, the method comprising,
a) orally administering to said animal a composition comprising plant or plant product comprising a S1-LTB fusion protein, wherein said S1-LTB fusion protein comprises the Spike (S1) protein of PEDV operably fused to the heat labile enterotoxin B subunit (LTB) peptide, wherein said S1 protein comprises SEQ ID NO: 3, 4, 9, 21 or 22 or a sequence having at least 90% or at least 95% identity to SEQ ID NO: 3, 4, 9, 21 or 22 or a functional fragment of said S1 protein; and wherein said LTB peptide comprises SEQ ID NO: 15 or a sequence having at least 90% or at least 95% identity to SEQ ID NO: 15 or a functional fragment of said LTB peptide; and
b) producing a mucosal immune response to said PEDV in said animal, wherein said mucosal immune response in said animal is greater in comparison to an animal that has been administered a composition comprising said S1 protein without said LTB peptide operably fused thereto.

8. The method of claim 7, wherein said mucosal immune response comprises an increase in fecal anti-PEDV immunoglobulin A (IgA), and wherein levels of fecal anti-PEDV IgA are higher in an animal administered a composition comprising said S1-LTB fusion protein in comparison to an animal administered a composition comprising said S1 protein without said LTB peptide operably fused thereto.

9. The method of claim 7, wherein said composition comprising said S1-LTB fusion protein comprises a higher ratio of high molecular weight S1 protein to low molecular weight S1 protein in comparison to a composition comprising said S1 protein without said LTB peptide operably fused thereto.

10. The method of claim 1, wherein milk of said animal comprises said S1-LTB fusion protein.

11. The method of claim 1, wherein said composition comprises plant material or plant tissue.

12. The method of claim 1, wherein said composition comprises seed or embryo of seed.

13. The method of claim 1, wherein said animal is a pig.

14. (canceled)

15. The method of claim 1, wherein said S1 protein comprises SEQ ID NO: 3, 4, 9, 21 or 22 or a functional fragment thereof.

16. (canceled)

17. The method of claim 1, wherein said LTB peptide comprises SEQ ID NO: 15.

18. The method of claim 1, wherein said S1-LTB fusion protein further comprises at least one of the COE peptide of SEQ ID NO: 12 and the DC3 peptide of SEQ ID NO: 13 operably fused thereto.

19. The method of claim 1, wherein said plant or plant product comprises a construct comprising a nucleic acid molecule encoding said S1-LTB fusion protein operably linked to:
a) a promoter preferentially directing expression to seed tissue of a plant; and
b) a nucleic acid molecule targeting expression of said S1-LTB fusion protein in the endoplasmic reticulum of said plant.

20. The method of claim 19, wherein said construct comprises two copies of said nucleic acid molecule encoding said S1-LTB fusion protein.

21. The method of claim 19, wherein said S1 protein is encoded by a nucleotide sequence comprising SEQ ID NO: 1, or 25, or a sequence having at least 90% or at least 95% identity to SEQ ID NO: 1, or 25.

22-23. (canceled)

24. The method of claim 19, wherein said LTB peptide is encoded by a nucleotide sequence comprising SEQ ID NO: 14, or a sequence having at least 90% or at least 95% identity to SEQ ID NO: 14.

25-26. (canceled)


SEQUENCE LISTING (25 sequences)

SEQ ID NO: 1 - DNA, 2238 bp, synthetic construct
  misc_feature 1..2238: note = Synthetic construct
  source 1..2238: mol_type = other DNA
atggcgaacaagcacctgagccttagcctcttcctcgtgctcctgggcctctccgcctccctcgcctccggcgtcacaaggtgctcggctaacaccaacttccgcaggttcttctccaagttcaacgtgcaggctcccgccgtggtggtgctcggcggctacctgccaatcggcgagaaccagggcgtgaattcaacgtggtactgcgcgggccagcaccccacggcctcgggcgtccacggcatcttcgtgagccacatccgcggcggccacggcttcgagatcggcatctcgcaggagcccttcgacccatcgggctaccagctctacctgcacaaggccacgaacggcaacacgaacgctaccgccaggctgaggatctgccagttcccctccatcaagaccctgggcccaaccgcgaacaacgacgtgaccacgggcaggaactgcctgttcaacaaggccatcccggcgcacatgtccgagcacagcgtcgtgggcatcacgtgggacaacgaccgcgtcaccgtgttcagcgacaagatctactacttctacttcaagaacgactggtccagggtcgccaccaagtgctacaacagcggcggctgcgcgatgcagtatgtgtacgagccgacctactacatgctgaacgtgacgtcggcgggcgaggacggcatcagctaccagccctgcacggccaactgcatcggctacgccgcgaacgtgttcgccacggagcccaacggccacatcccagagggcttctccttcaacaactggttcctcctgtccaacgacagcaccctggtccacggcaaggtcgtgagcaaccagccgctcctggtgaactgcctcctggcgatccccaagatctacggcctcggccagttcttctccttcaaccagacgatcgacggcgtctgcaacggcgcggccgtgcagagggctcccgaggcgctgaggttcaacatcaacgacacctccgtcatcctggcggagggcagcatcgtgctccacacggccctgggcacgaacttctccttcgtgtgcagcaactccagcaacccccacctcgccacgttcgccatccccctgggcgctacccaggtcccctactactgcttcctgaaggtggacacctacaacagcaccgtctacaagttcctggccgtgctgccgcccacggtccgcgagatcgtgatcaccaagtacggcgacgtctacgtgaacggcttcggctacctccacctgggcctcctggacgcggtcaccatcaacttcaccggccacggcacggacgacgacgtgtccggcttctggacgatcgccagcaccaacttcgtggacgccctgatcgaggtgcagggcaccgccatccagcgcatcctctactgcgacgacccggtgtcccagctgaagtgcagccaggtggcgttcgacctcgacgacggcttctaccccatctccagcaggaacctcctgtcccacgagcagccgatcagcttcgtcacgctcccctccttcaacgaccacagcttcgtcaacatcaccgtgtccgcttccttcggtggccactcgggcgctaacctgatcgccagcgacaccacgatcaacggcttctccagcttctgcgtggacacgaggcagttcaccatctccctcttctacaacgtcacgaacagctacggctacgtgtccaagagccaggactccaactgcccgttcaccctccagagcgtcaacgactacctgtccttcagcaagttctgcgtgtccacgagcctgctggcttcggcctgcaccatcgacctgttcggctaccccgagttcggctccggcgtcaagttcacgagcctctacttccagttcaccaagggcgagctcatcacgggcacccccaagccactggagggcgtcacggacgtgagcttcatgaccctggacgtgtgcaccaagtacacgatctacggcttcaagggcgagggcatcatcaccctcacgaactcaagctttctggccggcgtctactacacctccgacagcggccagctcctggccttcaagaacgtgacctcgggcgccgtctactcggtgacgccctgctccttcagcgagcaggctgcctacgtggacgacgacatcgtcggcgtgatctccagcctctcctcttcgactttcaactcaacccgcgagctgcccggcttcttctaccattccaaggacgagctctgatag

SEQ ID NO: 2 - DNA, 2415 bp, synthetic construct
  misc_feature 1..2415: note = Synthetic construct
  source 1..2415: mol_type = other DNA
atggcgaacaagcacctgagccttagcctcttcctcgtgctcctgggcctctccgcctccctcgcctccggcgtcacaaggtgctcggctaacaccaacttccgcaggttcttctccaagttcaacgtgcaggctcccgccgtggtggtgctcggcggctacctgccaatcggcgagaaccagggcgtgaattcaacgtggtactgcgcgggccagcaccccacggcctcgggcgtccacggcatcttcgtgagccacatccgcggcggccacggcttcgagatcggcatctcgcaggagcccttcgacccatcgggctaccagctctacctgcacaaggccacgaacggcaacacgaacgctaccgccaggctgaggatctgccagttcccctccatcaagaccctgggcccaaccgcgaacaacgacgtgaccacgggcaggaactgcctgttcaacaaggccatcccggcgcacatgtccgagcacagcgtcgtgggcatcacgtgggacaacgaccgcgtcaccgtgttcagcgacaagatctactacttctacttcaagaacgactggtccagggtcgccaccaagtgctacaacagcggcggctgcgcgatgcagtatgtgtacgagccgacctactacatgctgaacgtgacgtcggcgggcgaggacggcatcagctaccagccctgcacggccaactgcatcggctacgccgcgaacgtgttcgccacggagcccaacggccacatcccagagggcttctccttcaacaactggttcctcctgtccaacgacagcaccctggtccacggcaaggtcgtgagcaaccagccgctcctggtgaactgcctcctggcgatccccaagatctacggcctcggccagttcttctccttcaaccagacgatcgacggcgtctgcaacggcgcggccgtgcagagggctcccgaggcgctgaggttcaacatcaacgacacctccgtcatcctggcggagggcagcatcgtgctccacacggccctgggcacgaacttctccttcgtgtgcagcaactccagcaacccccacctcgccacgttcgccatccccctgggcgctacccaggtcccctactactgcttcctgaaggtggacacctacaacagcaccgtctacaagttcctggccgtgctgccgcccacggtccgcgagatcgtgatcaccaagtacggcgacgtctacgtgaacggcttcggctacctccacctgggcctcctggacgcggtcaccatcaacttcaccggccacggcacggacgacgacgtgtccggcttctggacgatcgccagcaccaacttcgtggacgccctgatcgaggtgcagggcaccgccatccagcgcatcctctactgcgacgacccggtgtcccagctgaagtgcagccaggtggcgttcgacctcgacgacggcttctaccccatctccagcaggaacctcctgtcccacgagcagccgatcagcttcgtcacgctcccctccttcaacgaccacagcttcgtcaacatcaccgtgtccgcttccttcggtggccactcgggcgctaacctgatcgccagcgacaccacgatcaacggcttctccagcttctgcgtggacacgaggcagttcaccatctccctcttctacaacgtcacgaacagctacggctacgtgtccaagagccaggactccaactgcccgttcaccctccagagcgtcaacgactacctgtccttcagcaagttctgcgtgtccacgagcctgctggcttcggcctgcaccatcgacctgttcggctaccccgagttcggctccggcgtcaagttcacgagcctctacttccagttcaccaagggcgagctcatcacgggcacccccaagccactggagggcgtcacggacgtgagcttcatgaccctggacgtgtgcaccaagtacacgatctacggcttcaagggcgagggcatcatcaccctcacgaactcaagctttctggccggcgtctactacacctccgacagcggccagctcctggccttcaagaacgtgacctcgggcgccgtctactcggtgacgccctgctccttcagcgagcaggctgcctacgtggacgacgacatcgtcggcgtgatctccagcctctcctcttcgactttcaactcaacccgcgagctgcccggcttcttctaccattccaacgatggctccaattgcacagagcctgtgttggtgtacagcaacatcggtgtttgcaaatctggcagcattggctacgtcccatcccagtctggccaagtgaagattgccccgaccgttactgggaacatcagcattcccaccaacttcagcttctacccctcctaccacagcaccccacagcgcccctgatag

SEQ ID NO: 3 - protein, 716 aa, Porcine epidemic diarrhea virus
VTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHS

SEQ ID NO: 4 - protein, 767 aa, Porcine epidemic diarrhea virus
VTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFS

SEQ ID NO: 5 - DNA, 72 bp, Hordeum vulgare
  source 1..72: mol_type = genomic DNA
atggcgaacaagcacctgagccttagcctcttcctcgtgctcctgggcctctccgcctccctcgcctccggc

SEQ ID NO: 6 - protein, 24 aa, Hordeum vulgare
MANKHLSLSLFLVLLGLSASLASG

SEQ ID NO: 7 - DNA, 756 bp, synthetic construct
  misc_feature 1..756: note = Synthetic construct
  source 1..756: mol_type = other DNA
atggcgaacaagcacctgagccttagcctcttcctcgtgctcctgggcctctccgcctccctcgcctccggcgtgtccaacggctcaatcccagtggacgaggtcatccagcacctccgcaactggaacttcacgtggaacatcatcctcaccatcctcctggtggtcctgcagtacggccactacaagtactccgccttcctgtacggcgtcaagatggcgatcctctggatcctctggcccctggtgctcgctctctccctgttcgacgcttgggccagcttccaggtgaactgggtcttcttcgccttctccatcctcatggcgtgcatcaccctcatgctgtggatcatgtacttcgtcaacagcatcaggctgtggaggaggacccactcctggtggagcttcaaccccgagacggacgctctcctgaccacctccgtcatgggcaggcaggtgtgcatccccgtcctgggcgccccaaccggcgtgaccctcacgctgctgtcgggcaccctcctggtggagggctacaaggtggctaccggcgtgcaggtcagccagctccccaacttcgtgaccgtcgccaaggcgaccacgacgatcgtgtacggcagggtgggcaggtcggtgaacgcttcctcgggcaccggctgggcgttctacgtgaggtccaagcacggcgactacagcgcggtgagcaacccctcgtcggtcctgactgattcggagaaggtcctgcacctcgtgtagtga

SEQ ID NO: 8 - protein, 250 aa, synthetic construct
  REGION 1..250: note = Synthetic construct
MANKHLSLSLFLVLLGLSASLASGVSNGSIPVDEVIQHLRNWNFTWNIILTILLVVLQYGHYKYSAFLYGVKMAILWILWPLVLALSLFDAWASFQVNWVFFAFSILMACITLMLWIMYFVNSIRLWRRTHSWWSFNPETDALLTTSVMGRQVCIPVLGAPTGVTLTLLSGTLLVEGYKVATGVQVSQLPNFVTVAKATTTIVYGRVGRSVNASSGTGWAFYVRSKHGDYSAVSNPSSVLTDSEKVLHLV

SEQ ID NO: 9 - protein, 713 aa, Porcine epidemic diarrhea virus
VTRCQSTINFRRFFSKFNVQAPAVVVLGGYLPSMNSSSWYCGTGIETDSGVHGIFLSYIDSGQGFEIGISQEPFDPSGYQLYLHKATNGNTNAIARLRICQFPDNKTLGPTVNDVTTGRNCLFNKAIPAYLQDGKNIVVGITWDNDRVTVFADKIYHFYLKNDWSRVATRCYNKRSCAMQYVYTPTYYMLNVTSAGEDGIYYEPCTANCSGYAANVFATDSNGHIPEGFSFNNWFLLSNDSTLLHGKVVSNQPLLVNCLLAIPKIYGLGQFFSFNQTMDGVCNGAAAQRAPEALRFNINDTSVILAEGSIVLHTALGTNLSFVCSNSSDPHLAIFAIPLGATEVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSAAFGGHSGANLIASDTTINGFSSFCVDTRQFTITLFYNVTNSYGYVSKSQDSNCPFTLQSVNDYLSFSKFCVSTSLLAGACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSNSTFNNTRELPGFFYHS

SEQ ID NO: 10 - DNA, 1392 bp, synthetic construct
  misc_feature 1..1392: note = Synthetic construct
  source 1..1392: mol_type = other DNA
atggcgaacaagcacctgagccttagcctcttcctcgtgctcctgggcctctccgcctccctcgcctccggcgtttccttccaggacaggggcaggaagcgcgtgcccctctcgctctacgcgcccctcagggtcaccaacgacaagccgctcagcaaggtgctggccaataacgcggtccccacgaacaagggcaataaggaccagcagatcggctactggaacgagcagatccggtggaggatgaggaggggcgagaggatcgagcagccctccaactggcacttctactacctcggcacgggcccacacgcggacctgaggtaccgcaccaggacggagggcgtgttctgggtggccaaggagggcgctaagacggagcccaccaatctcggcgtgcgcaaggcctcggagaagccgatcatccccaacttctcccagcagctgccgagcgtggtcgagatcgtggagccaaacaccccgcccacctcgcgcgctaattcccgcagcaggtcccgcggcaacggcaacaataggagccgctccccgagcaacaacaggggcaataaccagtcccgcggcaacagccagaaccggggcaataaccagggcaggggcgccagccagaacaggggcggcaataacaacaataacaacaagtcccgcaatcagagcaagaacaggaaccagtcgaatgaccgcggcggcgtgacctcgcgcgacgacctcgtggcggccgtcaaggacgcgctcaagtccctgggcatcggcgagaacccggacaagctgaagcagcagcagaagccaaagcaggagaggtcggactccagcggcaagaacaccccgaagaagaataagtcccgcgccacgagcaaggagagggacctgaaggacatccccgagtggcgcaggatcccgaagggcgagaactcggtggcggcctgcttcggcccccgcggcggcttcaagaatttcggcgacgcggagttcgtggagaagggcgtggacgcttcgggctacgctcagatcgccagcctggctccaaacgtggcggccctcctgttcggtggcaacgtggcggtcagggagctggcggacagctacgagatcacctacaactacaagatgacggtgccgaagtccgaccccaatgtcgagctcctggtgagccaggtcgacgccttcaagaccggcaacgccaagccgcagcgcaagaaggagaagaagaacaagagggagacgacgcagcagctcaatgaggaggccatctacgacgacgtgggcgtcccatcggacgtgacgcacgccaacctggagtgggacaccgctgtcgacggcggggatactgctgtggagatcatcaacgagattttcgatactgggaattagtga

SEQ ID NO: 11 - protein, 462 aa, synthetic construct
  REGION 1..462: note = Synthetic construct
MANKHLSLSLFLVLLGLSASLASGVSFQDRGRKRVPLSLYAPLRVTNDKPLSKVLANNAVPTNKGNKDQQIGYWNEQIRWRMRRGERIEQPSNWHFYYLGTGPHADLRYRTRTEGVFWVAKEGAKTEPTNLGVRKASEKPIIPNFSQQLPSVVEIVEPNTPPTSRANSRSRSRGNGNNRSRSPSNNRGNNQSRGNSQNRGNNQGRGASQNRGGNNNNNNKSRNQSKNRNQSNDRGGVTSRDDLVAAVKDALKSLGIGENPDKLKQQQKPKQERSDSSGKNTPKKNKSRATSKERDLKDIPEWRRIPKGENSVAACFGPRGGFKNFGDAEFVEKGVDASGYAQIASLAPNVAALLFGGNVAVRELADSYEITYNYKMTVPKSDPNVELLVSQVDAFKTGNAKPQRKKEKKNKRETTQQLNEEAIYDDVGVPSDVTHANLEWDTAVDGGDTAVEIINEIFDTGN

SEQ ID NO: 12 - DNA, 558 bp, synthetic construct
  misc_feature 1..558: note = Synthetic construct
  source 1..558: mol_type = other DNA
atggcgaacaagcacctgagccttagcctcttcctcgtgctcctgggcctctccgcctccctcgcctccggctcccacgagcagccgatcagcttcgtcacgctcccctccttcaacgaccacagcttcgtcaacatcaccgtgtccgcttccttcggtggccactcgggcgctaacctgatcgccagcgacaccacgatcaacggcttctccagcttctgcgtggacacgaggcagttcaccatctccctcttctacaacgtcacgaacagctacggctacgtgtccaagagccaggactccaactgcccgttcaccctccagagcgtcaacgactacctgtccttcagcaagttctgcgtgtccacgagcctgctggcttcggcctgcaccatcgacctgttcggctaccccgagttcggctccggcgtcaagttcacgagcctctacttccagttcaccaagggcgagctcatcacgggcacccccaagccactggagggcgtcacggacgtgttctacccctcctaccacagcaccccacagcgcccctgatag

SEQ ID NO: 13 - DNA, 36 bp, synthetic construct
  misc_feature 1..36: note = DC3 nucletotide sequence
  source 1..36: mol_type = other DNA
ttctacccctcctaccacagcaccccacagcgcccc

SEQ ID NO: 14 - DNA, 309 bp, Escherichia coli
  source 1..309: mol_type = genomic DNA
gccccgcagtccatcaccgagctctgctccgagtaccacaacacccagatctacaccatcaacgacaagatcctctcctacaccgagagcatggccggcaagcgcgagatggtgatcatcaccttcaagtccggcgccaccttccaggtggaggtgccgggctcccagcacatcgactcccagaagaaggccatcgagcgcatgaaggacaccctccgcatcacctacctcaccgagaccaagatcgacaagctctgcgtgtggaacaacaagaccccgaactccatcgccgccatcagcatggagaac

SEQ ID NO: 15 - protein, 103 aa, Escherichia coli
APQSITELCSEYHNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 16 - DNA, 3002 bp, Zea mays
  source 1..3002: mol_type = genomic DNA
cggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactacac

SEQ ID NO: 17 - DNA, 2520 bp, Zea mays
  source 1..2520: mol_type = genomic DNA
gcgctccctgacgctgtcttgggagagctgcaagatgagacactccatcccgcgcagccctgtcgtggcgtcctcctggatggacacctgcatcgctgtcgccctccaccaactcacctgaacgaagaatagaataaaaaatggagggagctgagggggcagtggttgcgctgtagggaggagagagaccgcgtcattataagactatctgcaaccgttacctctaaatttttccctctatatcattttttccccatattttcccccctattttttcatctcccgcaacggtttctcctaaatactccccctatatctcactaccactataaaatattattttttataccaactatcaattttttatctactaacaattactcgtggacccacagcacagtgtttaggagatgaacagtgacacgctatatctggggggagagagaaagaggccggcgcgtagggggcgccgtaggggcactgctgcggctgtagagtaccccctacacgccgcatgcaagggaagggggctgtcaggggggcaatgttgcgcatagcctaaagagcggatgaagcggcttgcaatttgcacgctggattcataaatagtgcatattactaaaaaaaagggtggggataggtatagagagtctattagagttgatctaagacccggtttatttcagattataatctgtccggattatataatccagcgcaaataatacagtaggtaaacaaacaactagattatgggttcagattatataatctaaaccccagattatgataatctcataatctcctcaagagtagcttattggagattattttggcaaaagacccactacccatggttatgtaaatagaaattataatatatatcatcttttttctcaccttaaataaacaaataagggtattgttgtctttatgaataatctacatttgtataatctaaactaccaaacaactacatctagattataatctggattatataatttaaattataatctagattatataatttataagctgaaacaacccggccctaaagcactatcgtatcacctatctgaaataagtcacgggtttcgaacgtccacttgcgtcgcacggaattgcatgtttcttgttggaagcatattcacgcaatctccacacataaaggtttatgtataaacttacatttagctcagtttaattacagtcttatttggatgcatatgtatggttctcaatccatataagttagagtaaaaaataagtttaaattttatcttaattcactccaacatatatggattgagtacaatactcatgtgcatccaaacaaactacttatattgaggtgaatttggatagaaattaaactaacttacacactaagccaatctttactatattaaagcaccagtttcaacgatcgtcccgcgtcaatattattaaaaaactcctacatttctttataatcaacccgcactcttataatctcttctctactactataataagagagtttatgtacaaaataaggtgaaattatgtataagtgttctggatattggttgttggctccatattcacacaacctaatcaatagaaaacatatgttttattaaaacaaaatttatcatatatcatatatatatatataaaccgtagcaatgcacgggcatataactagtgcaacttaatacatgtgtgtattaagatgaataagagggtatccaaataaaaaacttgttcgcttacgtctggatcgaaaggggttggaaacgattaaatctcttcctagtcaaaattgaatagaaggagatttaatctctcccaatccccttcgatcatccaggtgcaaccgtataagtcctaaagtggtgaggaacacgaaacaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacacc

SEQ ID NO: 18 - DNA, 6510 bp, synthetic construct
  misc_feature 1..6510: note = Synthetic construct
  source 1..6510: mol_type = other DNA
ggcgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcacgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcacgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactaca

SEQ ID NO: 19 - DNA, 12 bp, synthetic construct
  misc_feature 1..12: note = Endoplasmic reticulum signal sequence
  source 1..12: mol_type = other DNA
aaggacgagctc

SEQ ID NO: 20 - DNA, 28038 bp, Porcine epidemic diarrhea virus
  source 1..28038: mol_type = genomic DNA
acttaaaaagattttctatctacggatagttagctctttttctagactcttgtctactcaattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccttcctccggttccgtctgggggttgtgtggataactagttccgtctagtttgaaactagtaactgtcggctatggctagcaaccatgttacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccaaaaacatttgtggttggctgttattttctaactgtaattacttcctcgaagagttagagcttacttttggtcgtcgtggtggtaacatcgtgccagttgaccaatacatgtgtggcgctgacggtaaacctgttcttcaggaatccgaatgggagtatacagatttctttgctgactccgaagacggtcaactcaacattgctggtatcacttatgtgaaggcctggattgtagagcgatcggatgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattctcaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagcgcgctcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagttcatcgccgaagtgcctgagaagttggctgcggctgttacagtttttgtcaacttcttgaatgagctttttgagtctgcctgtgactgcttaaaggtcggaggtaaaacctttaacaaggttggctcttatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccctcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattacggcccatgaacaacaggatctgcaagggttcgtaaccacttgttgcacgatgtcaggttttgagtgttttatgcctataatcccacagtgtccagcagtgcttgaagagattgatggtggtagcatctggcggtcttttatcactggtcttaatacaatgtgggatttttgcaagcatcttaaagtcagctttggactagatggcattgttgtcactgtagcacgcaaatttaaacgacttggtgctctcttggcagaaatgtataacacttacctttcaactgtggtggaaaacttggtactggccggtgttagcttcaagtattatgccaccagtgtcccaaaaattgttttgggctgttgttttcacagtgttaaaagtgttcttgcaagtgccttccagattcctgtccaggcaggcgttgagaagtttaaagtcttccttaactgtgttcaccctgttgtaccacgtgtcattgaaacttcttttgtggaattagaagagacgacatttaaaccaccagcactcaatggtagtattgctattgttgatggctttgctttctattatgatggaacactatactatcccaccgatggtaatagcgttgttcctatctgctttaagaagaaaggtggtggtgatgtcaaattctctgatgaagtctctgttaaaaccattgacccagtttataaggtctcccttgaatttgagttcgagtctgagactattatggctgtgcttaataaggctgttggtaattgtatcaaggttacaggtggttgggacgatgttgttgagtatatcaatgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgataaggtggcagacgtggctaactctgagcctgaggatgacggtcttaatgtagctcctgaaacaaatgtagagtctgaagttgaggaagttgccgcaaccttgtcctttattaaagatacaccttccacagttactaaggatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgatggttgtgcttttcgtatgacgccaactttggaaccgttcccatatggtgcttgtgctcagtgtgctcaagttttgatgcacacttttaaaagtattgttggcaccggcatcttttgtcgagatactactgctctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcctgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcacttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgactggcggtgcattgcttgaagccataaatgtatttaaaaagagtaacaaaacagtgcctgctggcaactgtgttacttttgagtgtgcagatatgatttctattactatggtagtattgccatctgacggtgatgctaattatgacaaaaattatgcacgcgccgtcgtcaaggtatctaagcttaaaggcaagttattgcttgctgttggtgatgccatgttgtattccaagttgtcccacctcagcgtgttaggtttcgtatccacacctgatgatgtggagcgtttctacgcaaataagagtgtggttattaaagttactgaggatacacgtagtgttaagactgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtctaagtacattgttcctgctggttctgtcactattgaacgtgtcacgcatgacggttgttgttgtagtaagcgtgttgtcactgcaccagttgtgaatgctagcgtgttgaagcttggcgtcgaggatggtctttgtccacatggtcttaactacattgacaaagttgttgtagttaaaggtactacaattgttgtcaatgttggaaaacctgtagtggcaccatcgcacctctttcttaagggtgtttcctacacaacattcctagataatggtaacggtgttgccggccattatactgtttttgatcatgacactggtatggtgcatgatggagatgtttttgtaccaggtgatctcaatgtgtctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagtattttgggtctctgttttagggcctttcgtaagagggatgttaaagttctagctggtgtaccccaacgtactggtattatattgcgtaaaagtgtgcgctataatgcaaaggctttgggtgtcttcttcaagctaaaactttattggttcaaagttcttggtaagtttagtttgggtatttatgcattgtatgcattactattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacatacttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagataacaaatacacttgcaaagagtcacttaaaaattgtagcataatcacagactttattgtttttaacaataatggttccaatgtaaatcaggttaagaatgcatgtgtgtatttttcacagatgctttgtaaacctgttaagttagtggactcagcgttgttggccagtttgtctgttgattttggtgcaagcttacatagtgcttttgttagtgtgttgtcgaatagttttggcaaagacctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttctgaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagcttttttgattttagtactcaggttagcagtgatagtgattatgacttcaagtatattgagagtggccagttgaagacttttgacaatccacttagttgtgtgcataatgtctttagtaacttcgaccagtggcatgatgccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgaaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctccacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttaatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttaccattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttttttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacttgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgccatgaatgtagcaaagagcgaatttgaccgtgaggcttctactcagcgtaagcttgatagaatggcggaacaggctgcagcacagatgtacaaagaggcacgagcagttaataggaagtccaaagttgtaagtgctatgcattcactgctttttggtatgttgagacgtttggacatgtcttctgtagacaccattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcctggtaagctgaagcagcgctccattaaggcagaaggagatggcatagttggagaaggtaaggcactttacaataatgagggtggacgtacttttatgtatgctttcatctcggacaaaccggacctgcgtgtagtcaagtgggagttcgatggtggttgtaacactattgagctagaaccaccacgtaagttcttggtggattctcctaatggtgcacagatcaagtatctctactttgttcgtaaccttaacacgttacgtaggggtgctgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagcactgatatggcttatttaaacgagtacggggctctagtgcagctcgactagagccmtgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgccatctatggtaacgtttgtagaaaggatcttaccgagtatactatgatggatttgtgttacgctttacgtaactttgatgaaaacaattgcgatgttcttaagagcattttaattaaggtaggcgcttgtgaggagtcctacttcaataataaagtctggtttgaccctgttgaaaatgaagacattcatcgtgtctatgcattgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgttggattgatggtgttccactggtaactacagctggttatcattttaaacagttaggtatagtttggaacaatgacctcaacttacactctagcaggctctctattaacgaattactccagttttgtagtgatcctgcattgcttatagcatcatcaccagcccttgttgatcagcgtactgtttgcttttcagttgcagcgctaggtacaggtatgactaaccagactgttaaacctggccatttcaataaggagttttatgacttcttacttgagcaaggtttcttttctgagggctctgagcttactttaaagcacttcttctttgcacagaagggtgatgcagctgttaaggattttgactactataggtataatagacctactgttctggacatttgccaagctcgcgtcgtgtatcaaatagtgcaacgctattttgatatttacgaaggtggttgtatcactgctaaagaggtggttgttacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgcttttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggactacccaaagtgcgacagagcactgcccaatatgatacgtatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagtttttaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatgcagattgtcgataaagatggtacttattaccttccttaccctgatccttcaagaattctctctgcaggtgtgtttgttgatgacgttgttaaaactgatgcagttgtattgcttgaacgttatgtgtcattggctatagatgcctacccgttatctaagcatgaaaaccctgaatataagaaggtgttttatgtgcttttggattgggttaagcatctgtacaaaactcttaatgctggtgtgttagagtctttttctgtcacacttttggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaccacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctatgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcttccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtctacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttaggctttgacgttgaaggagcacatgttgttggctctaacgtcggtacaaatgtcccattgcaattagggttttctaacggtgttgattttgttgtcagacctgaaggttgcgttgtaacagagtctggtgactacattaaacccgtcagagctcgtgctccaccaggggaacaattcgcacaccttttgcctttacttaaacgcggccaaccatgggatgttgtccgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcatgtggcttctggtgatgccataatgactcgctgtctggccatacatgattgctttgtcaagaacgttgactggtccatcacatacccatttattggtaatgaggctgttattaataagagcggccgaattgtgcaatcacacactatgcggtcagttcttaagttatacaatccgaaagccatatatgatattggcaatcctaagggcattagatgtgccgtaacggatgctaagtggttttgctttgacaagaatcctactaattctaatgtcaagacattggagtatgactatataacacatggccaatttgatgggttgtgcttgttttggaattgcaatgtagacatgtatccagaattttctgtggtctgtcgttttgatactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggtgctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctccatgacccaaaatgctgtgcttatgtcacttacagctgttaaaaagcttactggcataaagttaacttatggttatcttaatggtgtcccagttaacacacatgaagataaaccttttacttggtatatttacactaggaagaacggcaagttcgaggaccatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttgcatctactaatttcgcaggtgcgtctggcctgtatgggtgtgctcaaaatagacgagtttgtgtctagtaatgatagcacgttaaagtcttgtactgttacatatgctgataaccctagtagtaagatggtttgtacgtatatggatctcctgcttgacgattttgtcagcattcttaaatctttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaggtttgagtattggacaatgttctgtaccagtgttaacacgtcatcgtcagaggcattcttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaacaggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattcctcaaatcctcacttagccaccttcgccatacctctgggtgctacccaagtaccttattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgcctaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtgatgtttcttggactttttcaatacacgattgacacagttgtcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgttctttattgcccacttttatattattgtggtgcatttttagatgcaactattatttgttgcacacttattggcaggctttgtttagtctgcttttactcctggcgctataaaaatgcgctctttattatttttaatactacgacactttctttcctcaatggtaaagcagcttattatgacggcaaatccattgtgattttagaaggtggtgaccattacatcacttttggcaactctcttgttgcttttgttagtagcatcgacttgtatctagctatacgtgggcggcaagaagctgacctacagctgttgcgaactgttgagcttcttgatggcaagaagctttatgtcttttcgcaacatcaaattgttggcattactaatgctgcatttgactcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccctagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggtccaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctataggactcgtactgagggtgttttctgggttgctaaagaaggcgcaaagactgaacccactaacctgggtgtcagaaaggcgtctgaaaagccaattattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaataaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggaatttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgccaatttggaatgggacacagctgttgatggtggtgacacggccgttgaaattatcaacgagatcttcgacacaggaaattaaacaatgtttgactggcttatcctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgactcaaggactgttagtaactgaagacctgacggtgttgatatggatacac

SEQ ID NO: 21 - protein, 1386 aa, Porcine epidemic diarrhea virus
MKSLTYFWLFLPVLSTLSLPQDVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPSDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQPYEVFEKVHVQ

SEQ ID NO: 22 - protein, 1386 aa, Porcine epidemic diarrhea virus
  VARIANT 728: note = X can be any naturally occurring amino acid
  VARIANT 905: note = X can be any naturally occurring amino acid
MRSLIYFWLSLPVLPTLSLPQDVYRCSARTNFRRFFSKFNVQAPAVVVLGGYLPSGETQGGAPRWYCAGRHETASGVHGIFLSHIRGGHGFEIGISQEPLDPSGYQLYLHKATNGNTNATARLRICQFPSNKTLGPTANDDVTTGRNCLFNKAIPAHMSEHSVVGITWDNDRVTVFADKIYHFYLKNEWSRVATKCYNSGGCAMQYVYEPIYYMLNVTSAGEDGISYQPCTANCIGYAANVFATESNGHIPEGFSFNNWFLLSNDSTLFHGKVVSNQPLLVNCLWAIPKIYGLGHFFSFNQTMDGVCNGATAYRAPEALRFNINDTSVILAEGSIVLHTALGTNLSFVCSNSSDPHKAIFSIPLGATQVPYYCFLKVDTYNSTVYKFLAVLPPTVREIVITKYGDVYVNGFGYLHLGLLDAVTINFTGHGTNDDVSGFWTIASTNFIDALVEVRATAIQRILYCDDPVCQLKCSQVSFDLDDGFYPISSRNLLSHEQPISFVTLPSFNDHSFVNITVSAAFGDSGGANLVASDTTINGFSSFCVDTRQFTIRLFYNVTSSYGYVSKSQDSNCPFTLQSVNDYLSFSKFCVSTSLLAGACTIDLFGYPAFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGGISSLSNSTFNNXRDFPGFFYHSNDGSNCTEPVLVYSNIGVCKSGSIGYVPLQDGQVKIAPTVIGNISIPTNFSMSIRTEYLQLYNTPVSVDCATYVCNGNSRCKQLLTQYTAACKTIESALELSARLESVEVNSMLTISEEALQLATISSFNGDGYNFTNVLGVSVYDSESGRVVHERSFIEDLLFNKXVTNGLGTVDEDYKRCSNGRSVADLVCAQYYSGIMVLPGVVDAEKLHMYSASLIGGMALGGFTAAVALPFSYAVQARLNYLALQTDVLQRNQQLLAESFNSAIGSITSAFESVNEAISQTSKGLNTVAHALTKVQEVVNSQGSALTQLTIQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVNVIAIAGLCVNGDIALTLREPGLVLFTHELQTHTATEYFVSSRRMFEPRKPTVSDFVQIQSCVVTYVNLTSDQLPDVIPDYVDVNKTLDEILASLPNRTGPNLPLDVFNATYLNLTGEIADLEQRSESLRNTTEELRSLIYNINNTLVDLEWLNRVETYIKWPWWVWLIVFIVLIFVVSLLVFCCISTGCCGCCGCCGACFSGCCRGPRLQPYEAFEKVHVQ

SEQ ID NO: 23 - protein, 1382 aa, Porcine epidemic diarrhea virus
MTPLIYFWLFLPVLLTLSLPQDVTRCQSTINFRRFFSKFNVQAPAVVVLGGYLPSMNSSSWYCGTGIETDSGVHGIFLSYIDSGQGFEIGISQEPFDPSGYQLYLHKATNGNTSAIARLRICQFPDNKTLGPTVNDVTTGRNCLFNKAIPALQDGKNIVVGITWDNDRVTVFADKIYHFYIKNDWSRVATRCYNKRSCAMQYVYTPTYYMLNVTSAGEDGIYYEPCTANCSGYAANVFATDSNGHIPEGFSFNNWFLLSNDSTLLHGKVVSNQPLLVNCLRAIPKIYGLGQFFSFNQTMDGVCNGAAAQRAPEALRFNINDTFVILAEGSIVLHTALGTNLSFVCSNSSDPHKAIFTIPLGVTEVPYYCFLKVDTYKSTVYKFLAVLPPTVKEIVITKYGDVYVNGFGYLHLGLLDAVTINFTGHGTDDDVSGFWTVASTNFVDALIEVQGTAIQRILYCDDPVSQLKCSQVSFDLDDGFYPISSRNLLSHEQPISFVTLPSFNDHSFVNITVSAAFGGHSGANLIASDTTTNGFSSFCVDTRQFTITLFYNVTNSYGYVSKSQDSNCPFTLQSVNDYLSFSKFCVSTSLLAGACTIDLFGYPEFGSGVKFTSLYFQFTKGELITGTPKPLQGVTDVSFMTLDVCTKYTIYGFKGEGIITLTNSSFLAGVYYTSDSGQLLAFKNVTSGAVYSVTPCSFSEQAAYVDDDIVGVISSLSNSTFNNTRELPGFFYHSNDGSNCTEPVLVYSNIGVCKSGSIGYVPLQDGQVKIAPMVTGNISIPTNFSMSIRTEYLQLYNTPVSVDCVTYVCNGNSRCKQLLTQYTAACKTIESALQLSARLESVEVNSMLTISEEALQLATISSFNGDGYNFTNVLGVSVYDPASGRVVQKGSFIEDLLFNKVVTNGLGTVDEDYKRCSNGRSVADLVCAQYYSGVMVLPGVVDAEKLHMYSASLLGGMALGGLTTAAALPFSNAVQARLNYLALQTDVLQRNQQLLAESFNSAIGNITSAFESVKEAISQTSNGLNTVAHALTKVQEVVKSQGSALTQLTIQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVNVIAIDGLCVNGDIALTLREPGLVLFTHELQTYTATEYFVSSRRMFEPRKPTVSDFVQIESCVVTYVNLTSDQLPDVIPDYIDVNKTLDEILASLPNRIGPSLPLDVFNATYLNLTGEIADLEQRSESLRNTTEELRSLIYNINNTLVDLEWLNRVETYIKWPWWVWLIIFIVLIFVVSLLVFCCISTGCCGCCGCCGACFSGCCRGPRLQPYEAFEKVHVQ

SEQ ID NO: 24 - protein, 4 aa, synthetic construct
  REGION 1..4: note = Endoplasmic reticulum signal sequence
HDEL

SEQ ID NO: 25 - DNA, 2217 bp, synthetic construct
  misc_feature 1..2217: note = Synthetic construct
  source 1..2217: mol_type = other DNA
atggcgaacaagcacctgagccttagcctcttcctcgtgctcctgggcctctccgcctccctcgcctccggcgtcactcgctgccagtcgaccatcaacttccgcaggttcttctccaagttcaacgtgcaggcccccgcggtggtggtgctcggcggctacctgccctccatgaattctagctcctggtactgcggcacgggcatcgagacggactcgggcgtgcacggcatcttcctcagctacatcgactccggccagggcttcgagatcggcatctcgcaggagcccttcgacccatcgggctaccagctctacctgcacaaggccaccaacggcaacacgaacgcgatcgcccgcctcaggatctgccagttcccggacaacaagaccctgggcccaaccgtcaacgacgtgaccaccggccgcaactgcctgttcaacaaggcgatcccggcctacctccaggacggcaagaacatcgtcgtgggcatcacgtgggacaacgacagggtcaccgtgttcgcggacaagatctaccacttctacctcaagaacgactggtcccgcgtcgcgaccaggtgctacaacaagcgcagctgcgccatgcagtatgtgtacaccccgacgtactacatgctcaacgtgacctcggcgggcgaggacggcatctactacgagccgtgcacggccaactgctcgggctacgcggccaacgtgttcgctaccgactccaacggccacatcccggagggcttcagcttcaacaactggttcctcctgagcaacgactccaccctcctgcacggcaaggtcgtgtccaaccagccgctcctggtcaactgcctcctggcgatccccaagatctacggcctgggccagttcttcagcttcaaccagacgatggacggcgtgtgcaacggcgcggccgctcagagggcccccgaggccctcaggttcaacatcaacgacacgagcgtcatcctggccgagggctccatcgtgctccacacggctctgggcacgaacctcagcttcgtgtgctccaacagctccgacccccacctggccatcttcgctatccccctgggcgccacggaggtcccctactactgcttcctgaaggtggacacctacaacagcacggtctacaagttcctggcggtgctgccgcccacggtcagggagatcgtgatcaccaagtacggcgacgtctacgtgaacggcttcggctacctccacctgggcctcctggacgcggtcaccatcaacttcaccggccacggcacggacgacgacgtgagcggcttctggacgatcgcgagcaccaacttcgtggacgctctcatcgaggtgcagggcaccgcgatccagaggatcctgtactgcgacgacccggtcagccagctcaagtgctcccaggtggccttcgacctggacgacggcttctaccccatcagctcccgcaacctcctgagccacgagcagccgatctccttcgtcaccctgcccagcttcaacgaccactccttcgtcaacatcacggtgagcgccgctttcggtggccactcgggcgccaacctcatcgcttccgacaccacgatcaacggcttcagctccttctgcgtggacacgcgccagttcaccatcacgctgttctacaacgtcaccaactcctacggctacgtgagcaagtcccaggactccaactgcccgttcaccctgcagagcgtcaacgactacctcagcttctccaagttctgcgtgagcacgtccctcctggccggcgcttgcaccatcgacctcttcggctaccccgagttcggcagcggcgtcaagttcacgtccctgtacttccagttcaccaagggcgagctgatcacgggcacccccaagccactggagggcgtcacggacgtgagcttcatgaccctcgacgtgtgcaccaagtacacgatctacggcttcaagggcgagggcatcatcaccctgacgaactcaagctttctcgccggcgtctactacaccagcgactccggccagctcctggcgttcaagaacgtgacctcgggcgcggtctactcggtgacgccctgcagcttctccgagcaggccgcctacgtggacgacgacatcgtcggcgtgatcagctccctgagcaactcaacattcaacaacactcgggagctgcctggcttcttctaccattcctgatag